(19) United States
(12) Patent Application Publication
(10) Pub. No.: US 2011/0009745 A1
(43) Pub. Date: Jan. 13, 2011

(54) Ultrasonically determining flow parameters of a fluid flowing through a passage, by using far-field analysis

(75) Inventors: Shahar Seifer, Mazkeret Batia (IL); Victor Steinberg, Rechovot (IL)

Correspondence Address: MARTIN D. MOYNIHAN d/b/a PRTSI, INC., P.O. BOX 16446, ARLINGTON, VA, 22215, US

(73) Assignee: Yeda Research And Development Co. Ltd., Rechovot (IL)

(21) Appl. No.: 12/311,692

(22) PCT Filed: Oct. 7, 2007
(86) PCT No.: PCT/IL07/01207

§ 371 Date: Sep. 1, 2010

Related U.S. Application Data
(60) Provisional application No. 60/850,266, filed on Oct. 10, 2006.

(51) Int. Cl. G06F 19/00; A61B 8/00

(52) U.S. Cl. 600/437; 702/48

(57) ABSTRACT

Ultrasonically determining flow parameters of a fluid (12) flowing through a passage (14), using far-field analysis. Includes: (a) acquiring near-field amplitude and phase change values of ultrasound waves transmitted (30) into, propagating through, and scattered (32) by, the flowing fluid; (b) determining far-field scattering amplitude distribution, A(θ, Δf), as two-dimensional function of scattering angle, θ, and Doppler frequency shift, Δf, from the acquired near-field amplitude and phase change values; and (c) determining flow parameters (peak velocity, velocity distribution, flow rate) of the flowing fluid, from the scattering amplitude distribution. Implementable using ‘clamp-on’ techniques including ultrasound wave transmitter and ultrasound wave detector array, clamped on, in an oppositely facing configuration, to the passage, for transmitting and detecting ultrasound waves propagating perpendicular to net flow direction of the flowing fluid. Applicable to different fluids (liquid or/and gas) flowing through different passages (channels, conduits, or ducts) of different types and sizes of processes.

### FIELD AND BACKGROUND OF THE INVENTION

The present invention relates to ultrasonically determining flow parameters of a fluid flowing through a passage, and more particularly, to a method for ultrasonically determining flow parameters of a fluid flowing through a passage, by using far-field analysis. The present invention is generally applicable for ultrasonically determining flow parameters (particularly, peak velocity, velocity distribution, and flow rate) of essentially any type or kind, and form, of fluid (liquid or/and gas) flowing through essentially any type or kind, and size, of passage (channel, conduit, or duct) of essentially any type or kind, and size (small scale, medium scale, large scale), of process.

Particular exemplary applications of the present invention are a homogeneous or inhomogeneous, single phase or multiple phase, particulate-free or particulate-containing, liquid, such as water, an organic solvent, or a petroleum based liquid, flowing through a passage (e.g., pipe, tube) of a medium or large scale process (e.g., a residential or commercial clean water or waste water distribution process, an industrial manufacturing process, or a petroleum based liquid transfer process), or, such as a biological liquid (e.g., blood, urine, water), flowing through a passage (e.g., vessel, duct, organ) of a small scale biological (e.g., human or animal) process. The present invention is generally applicable to a turbulent flowing fluid or a laminar flowing fluid (i.e., characterized by a high or low Reynolds number, respectively).

The inventive method is generally implementable by using various different types of equipment and hardware, and associated software, which are known for ultrasonically determining flow parameters of a fluid flowing through a passage. The inventive method is particularly implementable by using ‘clamp-on’ types of equipment and hardware, and associated software, which include an ultrasound wave transmitter and an ultrasound wave detector array, clamped on, in an oppositely facing configuration, to outer walls of a passage through which the fluid flows, and which operate by transmitting and detecting, respectively, ultrasound waves that propagate normal or perpendicular to the main or net flow direction of the flowing fluid, and are scattered by the flowing fluid.

Theories, principles, and practices thereof, and, related and associated applications and subjects thereof, relating to ultrasonically determining flow parameters of a fluid flowing through a passage (channel, conduit, or duct), are well known and taught about in the prior art, and currently practiced in a wide variety of numerous different fields and areas of technology. For the purpose of establishing the scope, meaning, and fields or areas of application, of the present invention, the following background includes selected definitions and exemplary usages of terminology which are relevant to, and used for, disclosing the present invention.

### Main Techniques of Ultrasonically Determining Flow Parameters of a Fluid Flowing Through a Passage (Channel, Conduit, or Duct)

In the field(s) encompassing or/and relating to ultrasonically determining flow parameters (particularly, peak velocity, velocity distribution, and flow rate) of a fluid (liquid or/and gas) flowing through a passage, three main categories of techniques (methods, and associated equipment) are well established and practiced: (a) techniques known as, and based on, time-of-flight, transit time difference, or propagation time difference, (b) techniques known as, and based on, transit time correlation, cross-correlation, or tag flow, and (c) techniques known as, and based on, sound Doppler velocimetry, or Doppler flow. Complete theoretical descriptions, details, explanations, examples, and applications, of each of these main categories of techniques, and related subjects and phenomena, are readily available in the technical literature [selected recent examples cited hereinbelow], for example, in the fields of physics, fluid mechanics, fluid dynamics, hydrodynamics, fluid flow technology, and ultrasound technology. Each of these main categories of techniques, and main limitations associated therewith, are briefly described hereinbelow.

### (a) Time-of-Flight, Transit Time Difference, or Propagation Time Difference, Techniques

These techniques are based on measuring and determining flow parameters (velocity, flow rate) of a fluid flowing through a passage, by measuring the difference in the time-of-flight, transit time, or propagation time, of relatively simple ultrasound waves propagating in the flowing fluid along the flow path between upstream and downstream directions along the passage.

More specifically, in these techniques, in general, typically, an upstream transducer transmits ultrasound waves, at a non-normal or skewed angle relative to the direction of flow, into the flowing fluid. The transmitted ultrasound waves are detected by a downstream transducer, and then transmitted back to the upstream transducer. Under conditions when the fluid is not flowing, the time taken by the upstream transmitted ultrasound waves to be detected by the downstream transducer is the same as the time taken by the downstream transmitted ultrasound waves to be detected by the upstream transducer. However, under conditions when the fluid is flowing, the effect of the flowing fluid velocity on the transmitted ultrasound waves is to effectively ‘speed up’ propagation of the upstream transmitted ultrasound waves in the upstream to downstream direction, and to effectively ‘slow down’ propagation of the downstream transmitted ultrasound waves in the downstream to upstream direction. Such speeding up and slowing down of the transmitted ultrasound waves generates or causes the difference in the time-of-flight, transit time, or propagation time, of the ultrasound waves propagating in the flowing fluid along the flow path between the upstream and downstream directions along the passage, which is used for determining the velocity, and flow rate, of the flowing fluid.

Among the main limitations of these techniques are that they (i) don't account for details of the flow distribution throughout the passage (channel, conduit, or duct, e.g., pipe, tube), which affect calibration of the flow rate and depend on varying conditions within the passage, (ii) depend upon the angle at which the ultrasound waves are transmitted through the passage wall and subsequently through the flowing fluid, which require knowledge of the refraction indices of both the passage wall and the fluid, (iii) sometimes involve mode conversion with shear vibrations in the passage, thus the forward and backward sound paths are not exactly the same, (iv) have an operational model based on geometrical acoustics, so inevitable processes, such as wave diffraction, are considered disadvantageous, and (v) by theory and definition, such techniques are totally inapplicable to transmitting, receiving, and measuring, ultrasound waves propagating in the direction normal or perpendicular to the main or net flow direction of the flowing fluid.

References [1, 2-10] contain recent exemplary teachings of main category (a)-time-of-flight, transit time difference, or propagation time difference, techniques, for determining flow parameters of a fluid flowing through a passage (channel, conduit, or duct).

### (b) Transit Time Correlation, Cross-Correlation, or Tag, Flow, Techniques

These techniques are based on measuring and determining flow parameters (velocity, flow rate) of a fluid flowing through a passage (channel, conduit, or duct), by measuring ultrasound waves propagating in the flowing fluid that (i) are scattered by internally existing or/and externally provided (seeded) substances (tags) (e.g., particles, droplets, or/and gas bubbles) moving in the flowing fluid, or/and, (ii) are similarly perturbed (phase modulated) by internally existing or/and externally caused flow fluctuations (tags) arising due to (velocity, pressure, thermal, concentration, or/and density) inhomogeneities or gradients, or/and turbulence, moving in the flowing fluid, along the flow path between upstream and downstream directions of the passage.

More specifically, characteristic or representative features contained in the measured scattered or/and perturbed ultrasound waves are correlated (typically, via a time correlator defined by a cross correlation function and an associated correlation coefficient) based on a delay time between measurements of such features, followed by calculating therefrom the average flow rate of the flowing fluid. By detecting a similar pattern of phase modulation, of the scattered or/and perturbed ultrasound waves, in the upstream and downstream paths at different times (i.e., the delay time), the velocity of the flowing fluid may be directly derived from the distance between the paths divided by the elapsed time (delay time) between occurrence of the correlated modulation patterns.

The main limitation of these techniques is that they are most suitable to applications involving turbulent flowing fluids, having a Reynolds number above about 4000, and are therefore inapplicable to determining relatively low velocities and low flow rates of fluids flowing through a passage.

References [1, 11-13] contain recent exemplary teachings of main category (b)-transit time correlation, cross-correlation, or tag flow, techniques, for determining flow parameters of a fluid flowing through a passage.

### (c) Sound Doppler Velocimetry, or Doppler Flow, Techniques

These techniques are based on measuring and determining flow parameters (velocity, flow rate) of a fluid flowing through a passage (channel, conduit, or duct), by measuring ultrasound waves propagating in the flowing fluid that are reflected (not scattered), thereby being (Doppler) frequency shifted, by internally existing or/and externally provided (seeded) substances (e.g., particles, droplets, or/and gas bubbles) moving in the flowing fluid, or/and, by internally existing or/and externally caused flow fluctuations arising due to (velocity, pressure, thermal, concentration, or/and density) inhomogeneities or gradients, or/and turbulence, moving in the flowing fluid, along the flow path between upstream and downstream directions of the passage.

More specifically, in these techniques, in general, typically, a transducer transmits ultrasound waves, at a non-normal or skewed angle relative to the direction of flow, into the flowing fluid, which are then reflected and detected by a transducer. The frequency of a reflected ultrasound wave is (Doppler) shifted relative to the frequency of the initially transmitted ultrasound wave. The (Doppler) frequency shift is proportional to the velocity of the reflecting substance moving in the flowing fluid, and thus proportional to the velocity of the flowing fluid, which is then used for calculating the flow rate of the flowing fluid. Sound Doppler velocimetry, or Doppler flow, techniques, typically include use of fast Fourier transform (FFT or fft) spectral analysis for filtering out non-flow related frequency shift noise (e.g., low frequency mechanical vibrations, or/and, high frequency electromagnetic radiation interference) from the detected (Doppler) frequency shifts of the reflected ultrasound waves.

Among the main limitations of these (strictly) Doppler techniques are that they (i) ordinarily require scattering objects (e.g., particles, droplets, or/and gas bubbles) in the flow, (ii) acquire the back scattering signal that is generally weaker than the forward scattering signal, (iii) are usually limited in accuracy, and (iv) by theory and definition, such (strictly) Doppler shift techniques (which are different from ‘transverse’ Doppler techniques based on spectral broadening, in which the ultrasound beams are focused at a certain point in the flow and the frequency broadening is detected instead of the Doppler shift, as discussed immediately following), are totally inapplicable to transmitting, receiving, and measuring, ultrasound waves propagating in the direction normal or perpendicular to the main or net flow direction of the flowing fluid.

References [1, 14-19] contain recent exemplary teachings of main category (c)-sound Doppler velocimetry, or Doppler flow, techniques, for determining flow parameters of a fluid flowing through a passage.

In addition to the preceding summarized (strictly) Doppler techniques, as indicated, relatedly, there are also ‘transverse’ Doppler techniques based on modulation of the ultrasound beam intensity. In such techniques, there is spatial variation of the ultrasound intensity (for example, in a focused ultrasound beam), such that the ultrasound beam which is backscattered from moving objects may change its spectrum following a change in the velocity of the moving objects. When a moving object passes over a modulated ultrasound beam, the backscattering intensity reflects the ultrasound beam intensity at the specific location of the moving object. If the backscattering signal is acquired from one moving object at a time (which usually requires a focused ultrasound beam), then theory predicts a spectral broadening of the backscattering signal that is proportional to the transverse motion of the object. If the ultrasound beam has a focal length F and a wavelength λ, and the moving object has a diameter W, and a velocity v at a direction θ relative to the direction of the ultrasound beam, then the detected frequency bandwidth, B_{d}, is calculated from the following equation:

$${B}_{d} = \frac{2}{λ} \frac{W}{F} v sin θ$$

Strictly at θ=90°, there is no way to infer about the direction of the motion of the moving object, since the signal reveals no Doppler shift at all. However, the magnitude of the velocity can be obtained by using the above equation.

Among the main limitations of ‘transverse’ Doppler techniques are that (i) the lateral dimension of the sampling volume must be very small, since a gradient in the flow velocity at the lateral direction introduces an artefactual contribution to the measurement, (ii) there is no distinguishing between left-right and right-left motion at θ=90° (as discussed above), (iii) there are sources of spectrum broadening other than the motion of the scattering object and the geometry of the ultrasound beam, which introduce inaccuracies to the measurements, (iv) there must be scattering objects in the fluid in order to obtain a backscattering signal, and the scattering objects must be spherical, otherwise additional error is introduced to the measurements, and (v) making measurements is problematic in a time dependent flow, such as blood flow.

References [24 and 25] contain recent exemplary teachings of ‘transverse’ Doppler techniques, for determining flow parameters of a fluid flowing through a passage.

Ultrasound Wave Scattering with Far-Field Analysis Based on Huygens' Principle: The Near-Field/Far-Field Transformation Technique

In the field(s) encompassing or/and relating to ultrasonically determining flow parameters of a fluid flowing through a passage (channel, conduit, or duct), separate from the preceding described three main categories of well established and practiced techniques, recently [20, 21], the inventors of the present invention developed a new technique, herein, referred to as the technique of ‘Ultrasound wave scattering with far-field analysis based on Huygens’ Principle', or more briefly, as the ‘Near-field/Far-field’ transformation technique, for studying ultrasound waves scattered by a laminar [20] or turbulent [21] fluid flowing through a passage.

Therein, as described by the present inventors, this technique is based on measuring near-field ultrasound waves (i.e., ultrasound waves in a region or zone near or close to the source of the ultrasound waves) propagating in a flowing fluid, followed by using a mathematical description of Huygens' Principle for evaluating and analyzing various flow parameters of the flowing fluid. The technique is based on analyzing the effect of scattering ultrasound waves by coupling of the scattered ultrasound waves with the flowing fluid, particularly involving ultrasound wave scattering induced by a component of the flow velocity and velocity gradient of the flowing fluid along the direction of the transmitted ultrasound waves. The technique further provides information on the flow energy spatial spectrum, the temporal spectrum of the square of vorticity, and a velocity profile in a specific case of axial-symmetric flow (vortex), of the flowing fluid.

Additionally, therein, as described by the present inventors, the mathematical description of Huygens' Principle is used for evaluating and analyzing the scattered ultrasound waves in the far-field (i.e., beyond, or outside of, the near-field region or zone) of the flowing fluid. The Near-field/Far-field transformation technique basically corresponds to ‘transforming’, ‘projecting’, ‘extrapolating’, or ‘mapping’, data and information (i.e., properties, characteristics, and behavior) of ultrasound waves which are transmitted, scattered, and detected, in the near-field region or zone, of a fluid flowing in and through a passage, from the near-field region or zone to the far-field region or zone, of the flowing fluid, and using the transformed, projected, extrapolated, or mapped, data and information for evaluating and analyzing flow parameters of the fluid flowing in and through the passage.

In the field of optics and propagation of electromagnetic radiation, the well known Huygens' Principle states that any point on a wave front of light may be regarded as the source of secondary waves and that the surface (envelope) that is tangent to the secondary waves can be used to determine the future position of the wave front. Alternatively, and equivalently, stated, the wave front of a propagating wave of light at any instant conforms to the surface (envelope) of spherical wavelets emanating from every point on the wave front at the prior instant (with the understanding that the wavelets have the same speed as the overall wave).

Regarding propagation of sound waves, and analysis thereof, and as a basis of justifying the use of Huygens' Principle (conventionally directed to optics and propagation of electromagnetic radiation) for studying propagation and scattering of ultrasound waves in flowing fluids, as stated by the present inventors [20], it has been shown [e.g., 22, 23] that in the approximation of a plane sound wave propagating through a velocity field with a continuous distribution of vorticity in a finite scattering domain there is a linear relation between the Fourier component of the scattering sound wave amplitude and the spatial and temporal Fourier transform of the vorticity component normal to the plane of the wave propagation in a far-field limit. Based on the preceding, the present inventors describe, and exemplify, application of a mathematical description of Huygens' Principle to near-field measurements of ultrasound waves scattered by a fluid flowing through a passage, for constructing a far-field scattering wave function, in terms of a near-field scattering wave function, of the scattered ultrasound waves, which is then used for evaluating and analyzing parameters of scattered ultrasound waves in the far-field region or zone.

The present inventors' Near-field/Far-field transformation technique was successfully applied to a laminar flowing fluid [20] and to a turbulent flowing fluid [21], for evaluating and analyzing the flow parameters (e.g., flow energy spatial spectrum, temporal spectrum of the square of vorticity, and velocity profile of a single vortex) thereof. Therein, as illustratively described by the present inventors, for obtaining near-field measurements of ultrasound waves scattered by the flowing fluid, a highly coherent finite width (pulsating) beam of ultrasound waves (sinusoidal, frequency in the range of between 0.2 MHz and 7 MHz, and pulse duration of 18 μs) was generated by a transducer type ultrasound wave emitter (constructed from a composite piezoelectric material or other appropriate material), and the ultrasound wave pulses were transmitted into, propagated through, and scattered by, the fluid flowing inside the bounds of a hydrodynamic flow cell having well defined geometrical dimensions and operating parameters. Amplitude and phase of the scattered ultrasound waves were detected by a linear detector array of 62 or 64 separate, but closely spaced apart, simultaneously and synchronously operative ultrasound wave detectors (facing opposite, across from, and lying in the same plane as, the ultrasound wave emitter), which were operative with two PC data acquisition cards, 62 or 64 lock-in amplifiers, and a corresponding number of pre-amplifiers, according to a heterodyne type scheme of data acquisition. Structure functions of the velocity and vorticity fields deduced, via the Near-field/Far-field transformation technique, from the detected scattered ultrasound waves, were found to closely agree with those empirically obtained from particle image velocimetry (PIV) measurements (using a 1 Mb CCD camera, 15 mJ pulse IR laser, and seeded particle markers) simultaneously made on the same hydrodynamic system.

The present inventors' Near-field/Far-field transformation technique of ‘Ultrasound wave scattering with far-field analysis based on Huygens' Principle’ significantly differs from the preceding described three main categories of well established and practiced techniques, by being based exclusively on scattering (i.e., not reflection) phenomena, encompassing the entire velocity field of scattered ultrasound waves propagating in a measuring plane defined by, and including, a same plane of the transmitting region or zone of an ultrasound wave transmitter and the detecting region or zone of an ultrasound wave detector array. Moreover, in contrast to the preceding described techniques, this technique is applicable to laminar or turbulent flowing fluids.

It is noted, however, that the present inventors' Near-field/Far-field transformation technique was initially developed primarily for evaluating and analyzing flow parameters (velocity and vorticity fields) of a fluid flowing through a passage, in the ‘same’ direction as that of the transmitted ultrasound waves, in a measuring plane defined by, and including, the transmitting region or zone of an ultrasound wave transmitter and the detecting region or zone of an ultrasound wave detector array. Accordingly, the Near-field/Far-field transformation technique, as initially developed, is limited by ordinarily being inapplicable for evaluating and analyzing flow parameters of a flowing fluid, in the direction ‘normal or perpendicular’ to the direction of the transmitted ultrasound waves, i.e., in the (longitudinal) direction of flow of the flowing fluid, in a measuring plane defined by, and including, the transmitting region or zone of an ultrasound wave transmitter and the detecting region or zone of an ultrasound wave detector array, which is of significant current interest in a wide variety of fluid flow applications.

Moreover, the present inventors' Near-field/Far-field transformation technique was initially developed for evaluating and analyzing flow parameters of a flowing fluid outside of the Doppler frequency shift region or zone, i.e., without involving use of Doppler frequency shift information and data. More specifically, the system defined by the flowing fluid scattering the transmitted ultrasound waves was considered as being in a quasi-stationary state, by neglecting all frequency shifts which were much smaller than the frequency of ultrasound waves.

In spite of the above described extensive teachings in the fields encompassing or/and relating to ultrasonically determining flow parameters (such as velocity and flow rate) of a fluid flowing through a passage (channel, conduit, or duct), and in view of the above described various significant limitations associated with such teachings, there is an on-going need for developing and practicing improved or/and new techniques for ultrasonically determining flow parameters of a fluid flowing through a passage.

There is thus a need for, and it would be highly advantageous to have a method for ultrasonically determining flow parameters (particularly, peak velocity, velocity distribution, and flow rate) of a fluid (liquid or/and gas) flowing through a passage, by using far-field analysis. Moreover, there is a need for such an inventive method which is generally applicable for ultrasonically determining flow parameters of essentially any type or kind, and form, of fluid (liquid or/and gas) flowing through essentially any type or kind, and size, of passage (channel, conduit, or duct) of essentially any type or kind, and size (small scale, medium scale, large scale), of process.

There is additional need for such an inventive method which is generally applicable to a turbulent flowing fluid or a laminar flowing fluid (i.e., characterized by a high or low Reynolds number, respectively). There is also need for such an inventive method which is generally implementable by using various different types of equipment and hardware, and associated software, which are known for ultrasonically determining flow parameters of a flowing fluid. There is also need for such an inventive method which is particularly implementable by using ‘clamp-on’ types of equipment and hardware, and associated software, which include an ultrasound wave transmitter and an ultrasound wave detector array, clamped on, in an oppositely facing configuration, to outer walls of a passage through which the fluid flows, and which operate by transmitting and detecting, respectively, ultrasound waves that propagate normal or perpendicular to the main or net flow direction of the flowing fluid, and are scattered by the flowing fluid.

### SUMMARY OF THE INVENTION

The present invention relates to a method for ultrasonically determining flow parameters of a fluid flowing through a passage, by using far-field analysis. The present invention is generally applicable for ultrasonically determining flow parameters (particularly, peak velocity, velocity distribution, and flow rate) of essentially any type or kind, and form, of fluid (liquid or/and gas) flowing through essentially any type or kind, and size, of passage (channel, conduit, or duct) of essentially any type or kind, and size (small scale, medium scale, large scale), of process.

Particular exemplary applications of the present invention are a homogeneous or inhomogeneous, single phase or multiple phase, particulate-free or particulate-containing, liquid, such as water, an organic solvent, or a petroleum based liquid, flowing through a passage (e.g., pipe, tube) of a medium or large scale process (e.g., a residential or commercial clean water or waste water distribution process, an industrial manufacturing process, or a petroleum based liquid transfer process), or, such as a biological liquid (e.g., blood, urine, water), flowing through a passage (e.g., vessel, duct, organ) of a small scale biological (e.g., human or animal) process. The inventive method is generally applicable to a turbulent flowing fluid or a laminar flowing fluid (i.e., characterized by a high or low Reynolds number, respectively).

The inventive method is generally implementable by using various different types of equipment and hardware, and associated software, which are known for ultrasonically determining flow parameters of a fluid flowing through a passage. The inventive method is particularly implementable by using ‘clamp-on’ types of equipment and hardware, and associated software, which include an ultrasound wave transmitter and an ultrasound wave detector array, clamped on, in an oppositely facing configuration, to outer walls of a passage through which the fluid flows, and which operate by transmitting and detecting, respectively, ultrasound waves that propagate normal or perpendicular to the main or net flow direction of the flowing fluid, and are scattered by the flowing fluid.

Thus, according to the present invention, there is provided a method for ultrasonically determining flow parameters of a fluid flowing through a passage, by using far-field analysis, the method comprising: (a) acquiring near-field amplitude and phase change values of ultrasound waves transmitted into, propagating through, and scattered by, the flowing fluid; (b) determining a far-field scattering amplitude distribution, as a two-dimensional function of scattering angle and Doppler frequency shift, from the acquired near-field amplitude and phase change values; and (c) determining the flow parameters of the flowing fluid, from the far-field scattering amplitude distribution.

According to further characteristics in preferred embodiments of the invention described below, the near-field amplitude and phase change values are acquired for a measuring plane defined by, and including, a same plane of a transmitting region or zone of an ultrasound wave transmitter device and a detecting region or zone of an ultrasound wave detector array device, wherein direction of the transmitted ultrasound waves is normal or perpendicular to main or net direction of flow of the flowing fluid.

According to further characteristics in preferred embodiments of the invention described below, the near-field amplitude and phase change values are acquired in a near-field region or zone characterized and defined by a near-field distance extending or spanning from (i) a position or location of a scatterer located within a scattering region or zone of the flowing fluid subjected to, and scattering, the ultrasound waves transmitted into, and propagating through, the flowing fluid by an ultrasound wave transmitter device, until (ii) a position or location of a detecting region or zone of an ultrasound wave detector array device detecting the scattered ultrasound waves.

According to further characteristics in preferred embodiments of the invention described below, the near-field region or zone is characterized and defined by relation or condition: b_{NF}≦d²/2λ, wherein the parameter b_{NF} is the near-field distance, the parameter d is smaller length of either (i) length of transmitting region or zone of the ultrasound wave transmitter device or (ii) length of the detecting region or zone of the ultrasound wave detector array device, and the parameter λ is wavelength of the transmitted or scattered ultrasound waves.

According to further characteristics in preferred embodiments of the invention described below, the scatterer is a feature or characteristic of, or within, the flowing fluid which scatters the transmitted ultrasonic waves propagating through the flowing fluid, thereby causing a change in velocity of the transmitted ultrasonic waves compared to velocity of the transmitted ultrasonic waves propagating through the flowing fluid which are not scattered by the feature or characteristic.

According to further characteristics in preferred embodiments of the invention described below, the feature or characteristic is an internally existing, or/and externally caused, flow fluctuation arising due to a velocity, pressure, thermal, concentration, or/and density inhomogeneity or gradient, or/and turbulence, moving in the flowing fluid.

According to further characteristics in preferred embodiments of the invention described below, the feature or characteristic is an internally existing, or/and externally provided, substance moving in the flowing fluid.

According to further characteristics in preferred embodiments of the invention described below, the ultrasound waves are in a form of a pulsed beam.

According to further characteristics in preferred embodiments of the invention described below, the pulsed beam has a pulse duration in a range of between about 1 cycle and about 1000 cycles.

According to further characteristics in preferred embodiments of the invention described below, the ultrasound waves have a frequency in a range of between about 20,000 cycles per second (20 kHz or 0.02 MHz) and about 20,000,000 cycles per second (20,000 kHz or 20 MHz).

According to further characteristics in preferred embodiments of the invention described below, the ultrasound waves have a frequency in a range of between about 100,000 cycles per second (100 kHz or 0.1 MHz) and about 7,000,000 cycles per second (7,000 kHz or 7 MHz).

According to further characteristics in preferred embodiments of the invention described below, the transmitted ultrasound waves are transmitted into the flowing fluid by a clamp-on type of ultrasound wave transmitter assembly, clamped onto an outside surface of the passage in a configuration such that the transmitted ultrasound waves are transmitted into the flowing fluid in a direction normal or perpendicular to main or net flow direction of the flowing fluid.

According to further characteristics in preferred embodiments of the invention described below, the ultrasound waves are detected by a clamp-on type of ultrasound wave detector array assembly, clamped onto an outside surface of the passage in a configuration such that the detected ultrasound waves are detected in a direction normal or perpendicular to main or net flow direction of the flowing fluid.

According to further characteristics in preferred embodiments of the invention described below, the ultrasound waves are detected by a clamp-on type of ultrasound wave detector array assembly, clamped onto an outside surface of the passage in a configuration oppositely facing, and aligned with, an ultrasound wave transmitter assembly, in a measuring plane defined by, and including, a transmitting region or zone of the ultrasound wave transmitter assembly and a detecting region or zone of the ultrasound wave detector array assembly.

According to further characteristics in preferred embodiments of the invention described below, the ultrasound waves are detected by an ultrasound wave detector array assembly including a linear array of at least six separated, linearly closely spaced apart, and positioned along a same axis, simultaneously and synchronously operative, transducer type ultrasound wave detectors/receivers.

According to further characteristics in preferred embodiments of the invention described below, the near-field amplitude and phase change values are acquired according to a heterodyne type scheme of data acquisition, or a direct analog to digital conversion type scheme of data acquisition.

According to further characteristics in preferred embodiments of the invention described below, the far-field scattering amplitude distribution is determined for a measuring plane defined by, and including, a same plane of a transmitting region or zone of an ultrasound wave transmitter device and a detecting region or zone of an ultrasound wave detector array device, wherein direction of the transmitted ultrasound waves is normal or perpendicular to main or net direction of flow of the flowing fluid.

According to further characteristics in preferred embodiments of the invention described below, the far-field scattering amplitude distribution is determined in a far-field region or zone characterized and defined by a far-field distance extending or spanning from (i) a position or location of a scatterer located within a scattering region or zone of the flowing fluid subjected to, and scattering, the ultrasound waves transmitted into, and propagating through, the flowing fluid by an ultrasound wave transmitter device, until (ii) a position or location located at or beyond a detecting region or zone of an ultrasound wave detector array device detecting the scattered ultrasound waves.

According to further characteristics in preferred embodiments of the invention described below, the far-field region or zone is characterized and defined by relation or condition: b_{FF}>>dt²/2λ, wherein the parameter b_{FF} is the far-field distance, the parameter d is smaller length of either (i) length of transmitting region or zone of the ultrasound wave transmitter device or (ii) length of the detecting region or zone of the ultrasound wave detector array device, and the parameter λ is wavelength of the transmitted or scattered ultrasound waves.

According to further characteristics in preferred embodiments of the invention described below, the far-field scattering amplitude distribution is determined for a far-field virtual propagation of the ultrasound waves transmitted into, propagating through, and scattered by, the flowing fluid, being represented by far-field virtual transmitted ultrasound waves, and far-field virtual scattered ultrasound waves, virtually propagating in a direction of a far-field virtual position or location located at a far-field virtual distance, at or beyond a detecting region or zone of an ultrasound wave detector array assembly detecting the scattered ultrasound waves.

According to further characteristics in preferred embodiments of the invention described below, step (b) includes transforming, projecting, extrapolating, or mapping, the acquired near-field amplitude and phase change values from a near-field region or zone to a far-field region or zone, of the flowing fluid.

According to further characteristics in preferred embodiments of the invention described below, step (b) is performed according to either a first case, based on using the acquired near-field amplitude and phase change values expressed in terms of time series in a time domain, or, a second case, based on using the acquired near-field amplitude and phase change values expressed in terms of frequency components in a frequency domain, wherein the first case and the second case differ according to order of using a Fourier transform procedure.

According to further characteristics in preferred embodiments of the invention described below, step (b) includes constructing a far-field scattering wave function in terms of a near-field scattering wave function, from the acquired near-field amplitude and phase change values.

According to further characteristics in preferred embodiments of the invention described below, the far-field scattering wave function is constructed according to a first case, for any given instant of time, in terms of a time domain, or, according to a second case, for any given frequency component, in terms of a frequency domain.

According to further characteristics in preferred embodiments of the invention described below, the far-field scattering wave function is based on application of a mathematical description of Huygens' Principle of optics to the acquired near-field amplitude and phase change values.

According to further characteristics in preferred embodiments of the invention described below, step (b) is based on, and includes, using the acquired near-field amplitude and phase change values expressed in terms of time series in a time domain, for constructing a far-field scattering wave function in terms of the time domain, which is then transformed, via using a Fourier transform procedure, from the time domain to a frequency domain.

According to further characteristics in preferred embodiments of the invention described below, normal or absolute values of the far-field scattering wave function are taken for obtaining the far-field scattering amplitude distribution.

According to further characteristics in preferred embodiments of the invention described below, step (b) is based on, and includes, transforming, via using a Fourier transform procedure, the acquired near-field amplitude and phase change values expressed in terms of time series in a time domain, from the time domain to a frequency domain, and using the acquired near-field amplitude and phase change values expressed in terms of the frequency domain, for constructing a far-field scattering wave function in terms of the frequency domain.

According to further characteristics in preferred embodiments of the invention described below, in step (c), the flow parameters are peak velocity, velocity distribution, and flow rate, of the flowing fluid.

According to further characteristics in preferred embodiments of the invention described below, step (c) includes determining a peak velocity of the flowing fluid, being value of the velocity of the flowing fluid corresponding to a peak magnitude in the distribution function of velocity component of the flowing fluid which is normal or perpendicular to direction of the transmitted ultrasound waves.

According to further characteristics in preferred embodiments of the invention described below, the peak velocity is defined by, and determined from, an equation including a term for a partial derivative of the Doppler frequency shift with respect to the scattering angle, and a term for wavenumber of the transmitted ultrasound waves, corresponding to a slope, in terms of an axis of the Doppler frequency shift, with respect to an axis of the scattering angle, of a best fitting line of a crest, or crest-like, shape or form, in a graphical plot of the far-field scattering amplitude distribution two-dimensional function.

According to further characteristics in preferred embodiments of the invention described below, the peak velocity is defined by, and determined from, an equation of form:

vₚₑₐₖk=(2π/k₀)(∂Δf/∂θ)|_{crest},

wherein the term (∂Δf/∂θ)|_{crest} is a partial derivative of the Doppler frequency shift, Δf, with respect to the scattering angle, θ, the term k₀ is wavenumber of the transmitted ultrasound waves, and the term π is symbol for pi, being mathematical constant number, 3.14, wherein the equation corresponds to a slope, in terms of axis of the Doppler frequency shift, Δf, with respect to axis of the scattering angle, θ, of a best fitting line of a crest, or crest-like, shape or form, in a graphical plot of the far-field scattering amplitude distribution two-dimensional function.

According to further characteristics in preferred embodiments of the invention described below, the equation corresponds to direction of the crest, or crest-like, shape or form, which is visually observable in the graphical plot of the far-field scattering amplitude distribution two-dimensional function.

According to further characteristics in preferred embodiments of the invention described below, the peak velocity is used for determining a velocity distribution of the flowing fluid.

According to further characteristics in preferred embodiments of the invention described below, the velocity distribution is determined in terms of a probability distribution function of the velocity component of the flowing fluid which is normal or perpendicular to direction of the transmitted ultrasound waves.

According to further characteristics in preferred embodiments of the invention described below, the crest, or crest-like, shape or form, in the graphical plot of the far-field scattering amplitude distribution is used for determining a velocity distribution of the flowing fluid.

According to further characteristics in preferred embodiments of the invention described below, the peak velocity and the probability distribution function are used for determining a flow rate of the flowing fluid.

According to further characteristics in preferred embodiments of the invention described below, the flow rate of the flowing fluid is determined in terms of (i) the peak velocity, (ii) the probability distribution function, (iii) value of cross-sectional area of the passage through which flows the fluid, and (iv) a statistical geometrical factor representing a function of geometrical characteristics and parameters relating to the flowing fluid, the passage, transmission of the ultrasound waves into the flowing fluid, and measurement of the scattered ultrasound waves.

According to further characteristics in preferred embodiments of the invention described below, the fluid is of a type or kind selected from the group consisting of a liquid, a gas, and any combination thereof.

According to further characteristics in preferred embodiments of the invention described below, the fluid is of a type or kind selected from the group consisting of a pure liquid, a solution of at least two miscible liquids, a mixture of at least two immiscible liquids, a pure gas, a mixture of at least two pure gases, and any combination thereof.

According to further characteristics in preferred embodiments of the invention described below, the fluid is of a form selected from the group consisting of homogeneous, inhomogeneous, single phase, multiple phase, particulate-free, particulate-containing, and any combination thereof.

According to further characteristics in preferred embodiments of the invention described below, the fluid is a turbulent flowing fluid or a laminar flowing fluid, characterized by a high or low Reynolds number, respectively.

According to further characteristics in preferred embodiments of the invention described below, the passage is a type, kind, or form, of channel, conduit, or duct, through and along which the flowing fluid passes or moves.

According to further characteristics in preferred embodiments of the invention described below, the passage is a pipe or tube through and along which the flowing fluid passes or moves.

According to further characteristics in preferred embodiments of the invention described below, the passage is a vessel, duct, or organ, of a small scale biological process, and the fluid is a biological liquid.

The present invention is implemented by performing procedures, steps, and sub-steps, in a manner selected from the group consisting of manually, semi-automatically, fully automatically, and a combination thereof, involving use and operation of system units, system sub-units, devices, assemblies, sub-assemblies, mechanisms, structures, components, and elements, and, peripheral equipment, utilities, accessories, and materials, in a manner selected from the group consisting of manually, semi-automatically, fully automatically, and a combination thereof. Moreover, according to actual procedures, steps, sub-steps, system units, system sub-units, devices, assemblies, sub-assemblies, mechanisms, structures, components, and elements, and, peripheral equipment, utilities, accessories, and materials, used for implementing a particular embodiment of the disclosed invention, the procedures, steps, and sub-steps, are performed by using hardware, software, or/and an integrated combination thereof, and the system units, sub-units, devices, assemblies, sub-assemblies, mechanisms, structures, components, and elements, and, peripheral equipment, utilities, accessories, and materials, operate by using hardware, software, or/and an integrated combination thereof.

In particular, software used for implementing the present invention includes operatively connected and functioning written or printed data, in the form of software programs, software routines, software sub-routines, software symbolic languages, software code, software instructions or protocols, software algorithms, or/and a combination thereof. In particular, hardware used for implementing the present invention includes operatively connected and functioning electrical, electronic or/and electromechanical system units, sub-units, devices, assemblies, sub-assemblies, mechanisms, structures, components, and elements, and, peripheral equipment, utilities, accessories, and materials, which may include one or more computer chips, integrated circuits, electronic circuits, electronic sub-circuits, hard-wired electrical circuits, or/and combinations thereof, involving digital or/and analog operations. Accordingly, the present invention is implemented by using an integrated combination of the just described software and hardware.

### BRIEF DESCRIPTION OF THE DRAWINGS

The present invention is herein described, by way of example only, with reference to the accompanying drawings. With specific reference now to the drawings in detail, it is stressed that the particulars shown are by way of example and for purposes of illustrative description of the preferred embodiments of the present invention only, and are presented in the cause of providing what is believed to be the most useful and readily understood description of the principles and conceptual aspects of the present invention. In this regard, no attempt is made to show structural details of the present invention in more detail than is necessary for a fundamental understanding of the invention, the description taken with the drawings making apparent to those skilled in the art how the several forms of the invention may be embodied in practice. In the drawings:

FIG. 1 is a (block type) flow diagram of the main steps or procedures of a preferred embodiment of the method for ultrasonically determining flow parameters of a fluid flowing through a passage, by using far-field analysis, in accordance with the present invention;

FIG. 2 is a schematic diagram illustrating an exemplary generalized preferred embodiment of a clamp-on system used for implementing the method of the present invention, in accordance with the present invention;

FIG. 3 is a schematic diagram illustrating an exemplary embodiment of the main characteristics and parameters of far-field ‘virtual propagation’ of transmitted ultrasound waves and scattered ultrasound waves, with respect to system 10 illustrated in FIG. 2, as relating to determining a far-field scattering amplitude distribution, A(θ, Δf), as a two-dimensional function of the scattering angle, θ, and Doppler frequency shift, Δf, from near-field amplitude and phase change values of the transmitted ultrasound waves, in accordance with the present invention;

FIGS. 4a and 4b are three-dimensional graphical presentations of exemplary results of implementing the method of the present invention, for ultrasonically determining flow parameters of a pure liquid water type of fluid flowing through a silicon rubber pipe type of passage, in accordance with Example 1 of the present invention; and

FIGS. 5a and 5b are graphical presentations of exemplary results of implementing the method of the present invention, for ultrasonically determining flow parameters of tap water type of fluid flowing through a painted steel pipe type of passage, in accordance with Example 2 of the present invention.

### DESCRIPTION OF THE PREFERRED EMBODIMENTS

The present invention relates to a method for ultrasonically determining flow parameters of a fluid flowing through a passage, by using far-field analysis. The present invention is generally applicable for ultrasonically determining flow parameters (particularly, peak velocity, velocity distribution, and flow rate) of essentially any type or kind, and form, of fluid (liquid or/and gas) flowing through essentially any type or kind, and size, of passage (channel, conduit, or duct) of essentially any type or kind, and size (small scale, medium scale, large scale), of process.

The method for ultrasonically determining flow parameters of a fluid flowing through a passage, by using far-field analysis, includes the following main steps or procedures: (a) acquiring near-field amplitude and phase change values of ultrasound waves transmitted into, propagating through, and scattered by, the flowing fluid; (b) determining a far-field scattering amplitude distribution, as a two-dimensional function of scattering angle and Doppler frequency shift, from the acquired near-field amplitude and phase change values; and (c) determining the flow parameters of the flowing fluid, from the far-field scattering amplitude distribution.

Particular exemplary applications of the present invention are a homogeneous or inhomogeneous, single phase or multiple phase, particulate-free or particulate-containing, liquid, such as water, an organic solvent, or a petroleum based liquid, flowing through a passage (e.g., pipe, tube) of a medium or large scale process (e.g., a residential or commercial clean water or waste water distribution process, an industrial manufacturing process, or a petroleum based liquid transfer process), or, such as a biological liquid (e.g., blood, urine, water), flowing through a passage (e.g., vessel, duct, organ) of a small scale biological (e.g., human or animal) process. The inventive method is generally applicable to a turbulent flowing fluid or a laminar flowing fluid (i.e., characterized by a high or low Reynolds number, respectively).

The inventive method is generally implementable by using various different types of equipment and hardware, and associated software, which are known for ultrasonically determining flow parameters of a fluid flowing through a passage. The inventive method is particularly implementable by using ‘clamp-on’ types of equipment and hardware, and associated software, which include an ultrasound wave transmitter and an ultrasound wave detector array, clamped on, in an oppositely facing configuration, to outer walls of a passage through which the fluid flows, and which operate by transmitting and detecting, respectively, ultrasound waves that propagate normal or perpendicular to the main or net flow direction of the flowing fluid, and are scattered by the flowing fluid.

The method for ultrasonically determining flow parameters of a fluid flowing through a passage (channel, conduit, or duct), by using far-field analysis, of the present invention, is based on using, and further extending, in a new and non-obvious manner, the inventors' initially developed ‘Near-field/Far-field’ transformation technique of evaluating and analyzing velocity and vorticity fields in spatial and temporal domains of a laminar or turbulent flowing fluid, as summarized hereinabove in the Background section. Specifically, the present inventors' initially developed ‘Near-field/Far-field’ transformation technique is further extended, and used herein as part of implementing the present invention, for evaluating and analyzing flow parameters of a fluid flowing through a passage, in the direction ‘normal or perpendicular’ to the direction of the transmitted ultrasound waves, i.e., in the (longitudinal) direction of flow of the flowing fluid, in a measuring plane defined by, and including, the transmitting region or zone of an ultrasound wave transmitter and the detecting region or zone of an ultrasound wave detector array, which is of significant current interest in a wide variety of different fluid flow applications.

A main aspect of novelty and inventiveness of the method of the present invention, in view of relevant prior art teachings, relates to sequentially combined performance of main steps or procedures (b) and (c). Namely, determining a far-field scattering amplitude distribution, as a two-dimensional function of scattering angle and Doppler frequency shift, from the acquired (measured) near-field amplitude and phase change values, followed by using the far-field scattering amplitude distribution for determining flow parameters (particularly, peak velocity, velocity distribution, and flow rate) of the flowing fluid.

As illustratively described hereinbelow, in accordance with the present invention, subsequent to transmission of ultrasound waves into the flowing fluid, the transmitted ultrasound waves formed therefrom, propagate through the flowing fluid in the direction normal or perpendicular to the main or net flow direction of the flowing fluid, and therefore, propagate through the flowing fluid in the direction normal or perpendicular to a longitudinal axis of the passage. Scattering of the transmitted ultrasound waves by the flowing fluid, in the form of scattered ultrasound waves, occurs within the flowing fluid which has a velocity component along the same direction of (the beam of) the transmitted ultrasound waves (i.e., normal or perpendicular to a longitudinal axis of the passage), and exit through a wall of the passage, and are detected by an ultrasound wave detector array assembly.

By performing Step (b) of the method of the present, the present inventors' initially developed ‘Near-field/Far-field’ transformation technique [20, 21] is further extended, and used herein, in a novel and inventive way, as part of implementing the present invention, for constructing the far-field scattering wave function as a two-dimensional function of scattering angle, and Doppler frequency shift (according to a first case, for any given instant of time, in terms of the time domain, or, alternatively, according to a second case, for any given frequency component, in terms of the frequency domain), from the near-field amplitude and phase change values acquired in Step (a). Step (b) ultimately results in determination of the far-field scattering amplitude distribution, as a two-dimensional function of the scattering angle, and Doppler frequency shift.

The far-field scattering amplitude distribution, determined in Step (b), is used for performing Step (c), for determining flow parameters (particularly, peak velocity, velocity distribution, and flow rate) of the flowing fluid, in the direction normal or perpendicular to the direction of the transmitted ultrasound waves, i.e., in a longitudinal direction of flow of the flowing fluid, in a measuring plane defined by, and including, the transmitting region or zone of an ultrasound wave transmitter device and the detecting region or zone of an ultrasound wave detector array device, which is of significant current interest in a wide variety of different fluid flow applications.

In addition to the present inventors' justification for initially developing, and applying, the Near-field/Far-field transformation technique [20, 21], a main limitation associated with prior art techniques for detecting scattering of ultrasound waves which are transmitted and scattered in the near-field region or zone of a fluid flowing in and through a passage, is that the amplitude of the scattered ultrasound waves attenuates (i.e., decreases) as a function of increasing scattering angle. Such amplitude is typically hidden or ‘buried’ within the amplitude of (the beam of) the transmitted ultrasound waves propagating through, and scattered by, the flowing fluid.

Only in the far-field region or zone, of the flowing fluid, is the amplitude of the far-field (virtual) scattered ultrasound waves, as a function of increasing scattering angle, separable from the amplitude of the far-field (virtual) transmitted ultrasound waves, where such ultrasound waves (virtually) propagate in the direction of a far-field (virtual) position or location located at a far-field (virtual) distance, at or beyond the detecting region or zone of an ultrasound wave detector array device detecting the scattered ultrasound waves.

It is to be understood that the present invention is not limited in its application to the details of the order or sequence, and number, of steps or procedures, and sub-steps or sub-procedures, of operation or implementation of the method, or to the details of type, composition, construction, arrangement, order, and number, of system units, system sub-units, devices, assemblies, sub-assemblies, mechanisms, structures, components, elements, and configurations, and, peripheral equipment, utilities, accessories, and materials, used for implementing the present invention, set forth in the following illustrative description, accompanying drawings, and examples, unless otherwise specifically stated herein. Accordingly, the present invention is capable of other embodiments and of being practiced or carried out in various ways. Although steps or procedures, sub-steps or sub-procedures, and system units, system sub-units, devices, assemblies, sub-assemblies, mechanisms, structures, components, elements, and configurations, and, peripheral equipment, utilities, accessories, chemical reagents, and materials, which are equivalent or similar to those illustratively described herein can be used for practicing or testing the present invention, suitable steps or procedures, sub-steps or sub-procedures, and system units, system sub-units, devices, assemblies, sub-assemblies, mechanisms, structures, components, elements, and configurations, and, peripheral equipment, utilities, accessories, and materials, are illustratively described and exemplified herein.

It is also to be understood that all technical and scientific words, terms, or/and phrases, used herein throughout the present disclosure have either the identical or similar meaning as commonly understood by one of ordinary skill in the art to which this invention belongs, unless otherwise specifically defined or stated herein. Phraseology, terminology, and, notation, employed herein throughout the present disclosure are for the purpose of description and should not be regarded as limiting.

For example, the phrase ‘ultrasound waves’ is used throughout the present disclosure. It is to be fully understood that the phrase ‘ultrasonic waves’ is entirely synonymous, and equivalent, thereto. Additionally, for example, herein, in the context of the field and art of the present invention, the following selected terminology is applicable for illustratively describing the present invention. The term ‘passage’ generally and equivalently refers to a type, kind, or form, of channel, conduit, or duct, through and along which a flowing fluid may pass or move. The term ‘channel’ generally and equivalently refers to a passage (as defined hereinabove) or a course through and along which a flowing fluid may pass or move. The term ‘conduit’ generally and equivalently refers to a channel (as defined hereinabove) through and along which a flowing fluid may pass or move. The term ‘duct’ generally and equivalently refers to a passage (as defined hereinabove), being tubular, or a channel (as defined hereinabove), being tubular, through and along which a flowing fluid may pass or move.

In general, such a passage (channel, conduit, or duct) of a flowing fluid may have walls which are fully closed, or at least partly closed (at least partly opened). In general, the cross section of a passage (channel, conduit, or duct) of a flowing fluid may be of essentially any geometrical shape or configuration, for example, tubular (i.e., cylindrical), rectangular, triangular, or a combination thereof.

The term ‘path’ generally and equivalently refers to the route or course along which a flowing fluid may pass or move through and along a passage (channel, conduit, or duct) (as defined hereinabove). In general, a flowing fluid moves through and along a passage (channel, conduit, or duct) in a directed manner, particularly in the direction along a longitudinal axis of the passage (channel, conduit, or duct).

It is to be fully understood that, unless specifically stated otherwise, the phrase ‘operatively connected’ is generally used herein, and equivalently refers to the corresponding synonymous phrases ‘operatively joined’, and ‘operatively attached’, where the operative connection, operative joint, or operative attachment, is according to a physical, or/and electrical, or/and electronic, or/and mechanical, or/and electro-mechanical, manner or nature, involving various types and kinds of hardware or/and software equipment and components. Moreover, all technical and scientific words, terms, or/and phrases, introduced, defined, described, or/and exemplified, in the above Background section, are equally or similarly applicable in the illustrative description of the preferred embodiments, examples, and appended claims, of the present invention. Additionally, as used herein, the term ‘about’ refers to ±10% of the associated value.

Steps or procedures, sub-steps or sub-procedures, and, equipment and materials, system units, system sub-units, devices, assemblies, sub-assemblies, mechanisms, structures, components, elements, and configurations, and, peripheral equipment, utilities, accessories, and materials, as well as operation and implementation, of exemplary preferred embodiments, alternative preferred embodiments, specific configurations, and, additional and optional aspects, characteristics, or features, thereof, of a method for ultrasonically determining flow parameters of a fluid flowing through a passage, by using far-field analysis, according to the present invention, are better understood with reference to the following illustrative description and accompanying drawings. Throughout the following illustrative description and accompanying drawings, same reference notation and terminology (i.e., numbers, letters, or/and symbols), refer to same components, elements, and parameters. In selected accompanying drawings, a reference xyz coordinate axis system is shown for indicating x, y, and z, directions relative to the components, elements, and parameters, drawn therein.

In the following illustrative description of the method of the present invention, included are main or principal steps or procedures, and sub-steps or sub-procedures, and, main or principal system units, system sub-units, devices, assemblies, sub-assemblies, mechanisms, structures, components, elements, and configurations, and, peripheral equipment, utilities, accessories, and materials, needed for sufficiently understanding proper ‘enabling’ utilization and implementation of the disclosed invention. Accordingly, description of various possible preliminary, intermediate, minor, or/and optional, steps or procedures, or/and sub-steps or sub-procedures, or/and, system units, system sub-units, devices, assemblies, sub-assemblies, mechanisms, structures, components, elements, and configurations, and, peripheral equipment, utilities, accessories, and materials, of secondary importance with respect to enabling implementation of the invention, which are readily known by one of ordinary skill in the art, or/and which are available in the prior art and technical literature relating to the fields of the present invention, are at most only briefly indicated herein.

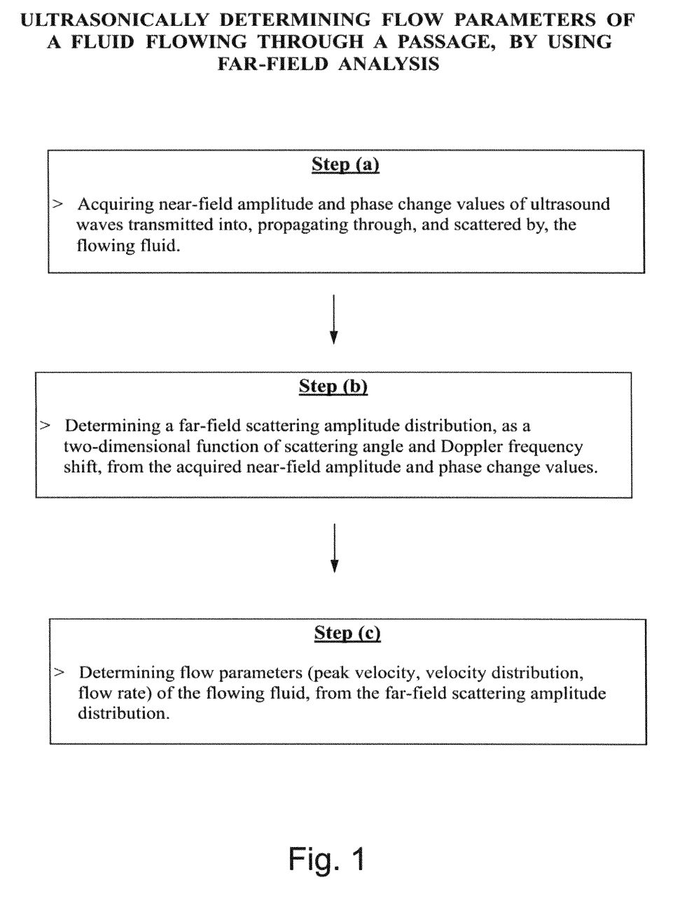

Referring now to the drawings, FIG. 1 is a (block type) flow diagram of the main steps or procedures of a preferred embodiment of the method for ultrasonically determining flow parameters of a fluid flowing through a passage, by using far-field analysis, of the present invention. In FIG. 1, each generally applicable, main step or procedure of the method of the present invention is enclosed inside a frame (block). Phraseology, terminology, and, notation, appearing in the following illustrative description are consistent with those appearing in the flow diagram illustrated in FIG. 1.

According to a main aspect of the present invention, as shown in FIG. 1, there is provision of a method for ultrasonically determining flow parameters of a fluid flowing through a passage, by using far-field analysis, the method including the following main steps or procedures: (a) acquiring near-field amplitude and phase change values of ultrasound waves transmitted into, propagating through, and scattered by, the flowing fluid; (b) determining a far-field scattering amplitude distribution, as a two-dimensional function of scattering angle and Doppler frequency shift, from the acquired near-field amplitude and phase change values; and (c) determining the flow parameters of the flowing fluid, from the far-field scattering amplitude distribution.

The method of the present invention is generally applicable for ultrasonically determining flow parameters (particularly, peak velocity, velocity distribution, and flow rate) of essentially any type or kind, and form, of fluid (liquid or/and gas) flowing through essentially any type or kind, and size, of passage (channel, conduit, or duct) of essentially any type or kind, and size (small scale, medium scale, large scale), of process. The inventive method is generally implementable by using various different types of equipment and hardware, and associated software, which are known for ultrasonically determining flow parameters of a fluid flowing through a passage. The inventive method is particularly implementable by using ‘clamp-on’ types of equipment and hardware, and associated software, which include an ultrasound wave transmitter and an ultrasound wave detector array, clamped on, in an oppositely facing configuration, to outer walls of a passage through which the fluid flows, and which operate by transmitting and detecting, respectively, ultrasound waves that propagate normal or perpendicular to the main or net flow direction of the flowing fluid, and are scattered by the flowing fluid.

### Generalized System Suitable for Determining Flow Parameters of a Flowing Fluid by Using Far-field Analysis

Immediately following is illustrative description of an exemplary generalized preferred embodiment of a clamp-on type of system, which is suitable for implementing the method for ultrasonically determining flow parameters of a flowing fluid by using far-field analysis, of the present invention (FIG. 1). Following hereafter is a further detailed illustrative description of the steps of the method of the present invention.

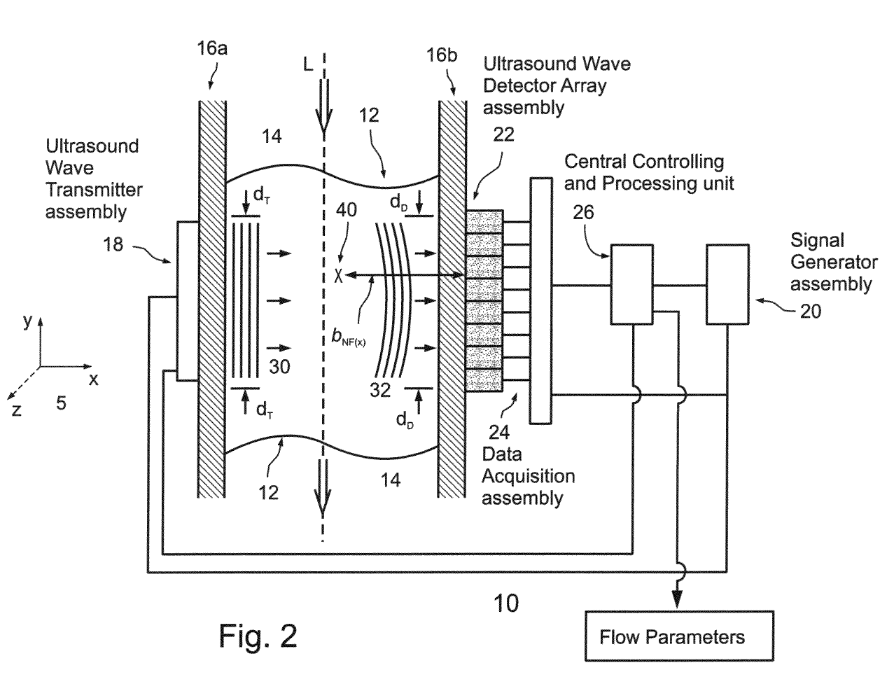

FIG. 2 is a schematic diagram illustrating an exemplary generalized preferred embodiment of a clamp-on type of system, herein, generally referred to as system 10, suitable for implementing the method of the present invention (FIG. 1). In FIG. 2, a reference xyz coordinate axis system 5, is shown for indicating x, y, and z, directions relative to the components, elements, and parameters, drawn therein. As shown in FIG. 2, a fluid 12 flows through a passage 14 which has walls 16a and 16b, and a (y-axis direction) longitudinal axis L (in FIG. 2, indicated by the dashed line drawn lengthwise along the y-axis direction through fluid 12 and passage 14). Fluid 12 flows through passage 14 in an essentially single direction (in FIG. 1, indicated by the double-tailed, single-headed, arrows drawn immediately before and after fluid 12, and coaxial with longitudinal axis L of passage 14).

Fluid 12 is, in general, essentially any type or kind, and form, of fluid. Exemplary types or kinds of fluid 12 are selected from the group consisting of a liquid, a gas, and any combination thereof. Additional exemplary types or kinds of fluid 12 are selected from the group consisting of a pure liquid, a solution of at least two miscible liquids, a mixture of at least two immiscible liquids, a pure gas, a mixture of at least two pure gases, and any combination thereof. Exemplary forms of fluid 12 are selected from the group consisting of homogeneous, inhomogeneous, single phase, multiple phase, particulate-free, particulate-containing, and any combination thereof. Fluid 12 is either a turbulent flowing fluid or a laminar flowing fluid, characterized by a high or low Reynolds number, respectively.

Passage 14 is, in general, a type, kind, or form, of channel, conduit, or duct, through and along which flowing fluid 12 passes or moves. As a duct, passage 14 is tubular in shape or form, through and along which flowing fluid 12 passes or moves. In general, passage (channel, conduit, or duct) 14 of flowing fluid 12 has walls 16a and 16b which are fully closed, or at least partly closed (at least partly opened). In general, the cross section of passage (channel, conduit, or duct) 14 of flowing fluid 12 may be of essentially any geometrical shape or configuration, for example, tubular (i.e., cylindrical), rectangular, triangular, or a combination thereof. As indicated, flowing fluid 12 moves through and along passage (channel, conduit, or duct) 14 in a directed manner, particularly in the main or net direction along, and coaxial with, a longitudinal axis, for example, (y-axis direction) longitudinal axis L, of passage (channel, conduit, or duct) 14.

Particular exemplary applications of the method of the present invention are wherein fluid 12 is a turbulent or laminar, homogeneous or inhomogeneous, single phase or multiple phase, particulate-free or particulate-containing, liquid, such as water, an organic solvent, or a petroleum based liquid, flowing through a pipe or tube type of passage 14 of a medium or large scale process (e.g., a residential or commercial clean water or waste water distribution process, an industrial manufacturing process, or a petroleum based liquid transfer process), or, such as a biological liquid (e.g., blood, urine, water), flowing through a vessel, duct, or organ, type of passage 14 of a small scale biological (e.g., human or animal) process.

As shown in FIG. 2, system 10, used for implementing the method of the present invention, for ultrasonically determining flow parameters of flowing fluid 12 by using far-field analysis, includes the following main components: an ultrasound wave transmitter assembly 18, a signal generator assembly 20, an ultrasound wave detector array assembly 22, a data acquisition assembly 24, and a central controlling and processing unit 26.

Ultrasound wave transmitter assembly 18 is for transmitting ultrasound waves, preferably, in the form of a pulsed beam, into and through wall 16a of passage 14, and, into flowing fluid 12, for forming (a pulsed beam of) transmitted ultrasound waves, for example, transmitted ultrasound waves 30.

Ultrasound wave transmitter assembly 18 is, preferably, a clamp-on type of ultrasound wave transmitter assembly, which is clamped (using clamping, adhering, or/and mounting, techniques well known in the art) onto the outside surface of wall 16a of passage 14, in a configuration such that ultrasound wave transmitter assembly 18 transmits (the pulsed beam of) ultrasound waves into and through wall 16a of passage 14, and, into flowing fluid 12, in the (x-axis) direction normal or perpendicular to the main or net flow (y-axis) direction of flowing fluid 12. Since flowing fluid 12 moves through and along passage 14 in a directed manner, particularly in the main or net (y-axis) direction along, and coaxial with, longitudinal axis L of passage 14, therefore, ultrasound wave transmitter assembly 18 transmits (the pulsed beam of) ultrasound waves into and through wall 16a of passage 14, and, into fluid 12, in the (x-axis) direction normal or perpendicular to longitudinal axis L of passage 14, for forming (a pulsed beam of) transmitted ultrasound waves, for example, transmitted ultrasound waves 30.

Ultrasound wave transmitter assembly 18 is, preferably, suitable for transmitting (a pulsed beam of) ultrasound waves having a frequency, preferably, in a range of between about 20,000 cycles per second (20 kHz or 0.02 MHz) and about 20,000,000 cycles per second (20,000 kHz or 20 MHz), and more preferably, in a range of between about 100,000 cycles per second (100 kHz or 0.1 MHz) and about 7,000,000 cycles per second (7,000 kHz or 7 MHz). Preferably, ultrasound wave transmitter assembly 18 transmits (the pulsed beam of) ultrasound waves according to a sinusoidal pulse, with a pulse duration in a range of, preferably, between about 1 cycle and about 1000 cycles, and more preferably, between about 10 cycles and about 100 cycles. In terms of time units, ultrasound wave transmitter assembly 18 transmits (the beam of) ultrasound waves according to a sinusoidal pulse, with a pulse duration in a range of between about 1 microsecond (μs) and about 1 millisecond (ms). The lower limit of the pulse duration depends upon the frequency of the transmitted (beam of) ultrasound waves, while the upper limit of the pulse duration depends upon the maximum velocity of flowing fluid 12.

For functioning (operating) as described hereinabove, ultrasound wave transmitter assembly 18 includes, for example, a transducer type ultrasound wave emitter, constructed, for example, from a piezoelectric material (e.g., a piezoelectric ceramic based composite material, such as a PZT (lead zirconate titanate) ceramic based composite material), or other appropriate material exhibiting vibrating type of transducer properties, characteristics, and behavior, suitable for emitting a pulsed beam of ultrasound waves. Such a transducer type ultrasound wave emitter typically includes a pair of electrodes for applying an electric voltage to the piezoelectric material, whereby the electric voltage is converted into a pulsed beam of ultrasound waves which is subsequently transmitted into and through wall 16a of passage 14, and, into flowing fluid 12, in the (x-axis) direction normal or perpendicular to the main or net flow (y-axis) direction of flowing fluid 12, for forming (a pulsed beam of) transmitted ultrasound waves, for example, transmitted ultrasound waves 30.

For the purpose of decreasing the difference in impedance existing between the outer surface of the transmitting region or zone of ultrasound wave transmitter assembly 18 and the outside surface of wall 16a of passage 14, and therefore, for increasing the strength or intensity of the (pulsed beam of) ultrasound waves transmitted into and through wall 16a of passage 14, and, into flowing fluid 12, optionally, a coupling material or substance (not shown in FIG. 2), such as a petroleum based gel or cream, or even water, is placed or applied between the outer surface of the transmitting region or zone of ultrasound wave transmitter assembly 18 and the outside surface of wall 16a of passage 14.

Signal generator assembly 20 is for generating a signal having an appropriate set of parameters which drives or controls the type of ultrasound waves transmitted by ultrasound wave transmitter assembly 18. Signal generator assembly 20 is, preferably, suitable for generating, and sending, a, preferably, pulsed, signal having an appropriate set of parameters which drives or controls ultrasound wave transmitter assembly 18 for transmitting ultrasound waves, preferably, in the form of a pulsed beam, into and through wall 16a of passage 14, and, into flowing fluid 12, for forming (a pulsed beam of) transmitted ultrasound waves, for example, transmitted ultrasound waves 30. Signal generator assembly 20 is, preferably, suitable for generating, and sending, a signal having an appropriate set of parameters which drives or controls ultrasound wave transmitter assembly 18 for transmitting (a pulsed beam of) ultrasound waves according to a sinusoidal pulse, with a pulse duration in a pre-determined range, as described hereinabove.

Ultrasound wave detector array assembly 22 is for detecting (the pulsed beam of) transmitted ultrasound waves 30 which propagate through, and are scattered by flowing fluid 12, as (a pulsed beam of) scattered ultrasound waves, for example, scattered ultrasound waves 32, which exit through wall 16b of passage 14. More specifically, ultrasound wave detector array assembly 22 is for detecting the (pulsed beam of) ultrasound waves transmitted by ultrasound wave transmitter assembly 18 into and through wall 16a of passage 14, and, into flowing fluid 12, which subsequently become transmitted ultrasound waves 30 propagating through, and scattered by, flowing fluid 12, in the form of scattered ultrasound waves 32, which exit through wall 16b of passage 14.

Subsequent to the preceding illustratively described transmission of ultrasound waves into flowing fluid 12, transmitted ultrasound waves 30 formed therefrom, propagate through flowing fluid 12 in the same (x-axis) direction normal or perpendicular to the main or net flow (y-axis) direction of flowing fluid 12, and therefore, propagate through flowing fluid 12 in the same direction normal or perpendicular to longitudinal axis L of passage 14 (in FIG. 2, indicated by the set of three arrows adjacent transmitted ultrasound waves 30, pointing in the (x-axis) direction towards, and normal or perpendicular to, longitudinal axis L of passage 14). Transmitted ultrasound waves 30 propagating through, and scattered by, flowing fluid 12, in the form of scattered ultrasound waves 32 (in FIG. 2, indicated by the set of three arrows adjacent scattered ultrasound waves 32, pointing in the (x-axis) direction away, and normal or perpendicular to, longitudinal axis L of passage 14), exit through wall 16b of passage 14, and are detected by ultrasound wave detector array assembly 22.

Ultrasound wave detector array assembly 22 is, preferably, designed, constructed, and operative, for being complementary to (i.e., simultaneously and synchronously operative with) ultrasound wave transmitter assembly 18. Accordingly, ultrasound wave detector array assembly 22 is, preferably, a clamp-on type of ultrasound wave detector array assembly, which is clamped (using clamping, adhering, or/and mounting, techniques well known in the art) onto the outside surface of wall 16b of passage 14, in a configuration such that ultrasound wave detector array assembly 22 detects (the pulsed beam of) transmitted ultrasound waves 30 which propagate through, and are scattered by flowing fluid 12, as (a pulsed beam of) scattered ultrasound waves 32, which exit through wall 16b of passage 14, in the (x-axis) direction normal or perpendicular to the main or net flow direction of flowing fluid 12. Ultrasound wave detector array assembly 22 is, preferably, clamped onto the outside surface of wall 16b of passage 14, in a configuration oppositely facing, and aligned with, ultrasound wave transmitter assembly 18, in a measuring plane defined by, and including, the transmitting region or zone of ultrasound wave transmitter assembly 18 and the detecting region or zone of ultrasound wave detector array assembly 22, wherein the (x-axis) direction of transmitted ultrasound waves 30 is normal or perpendicular to the main or net (y-axis) longitudinal direction of flowing fluid 12.

Ultrasound wave detector array assembly 22 is, preferably, suitable for detecting (a pulsed beam of) scattered ultrasound waves 32 having a frequency, preferably, in a range of between about 20,000 cycles per second (20 kHz or 0.02 MHz) and about 20,000,000 cycles per second (20,000 kHz or 20 MHz), and more preferably, in a range of between about 100,000 cycles per second (100 kHz or 0.1 MHz) and about 7,000,000 cycles per second (7,000 kHz or 7 MHz). Preferably, ultrasound wave detector array assembly 22 detects (the pulsed beam of) scattered ultrasound waves 32 according to a sinusoidal pulse, with a pulse duration in a range of, preferably, between about 1 cycle and about 1000 cycles, and more preferably, between about 10 cycles and about 100 cycles. In terms of time units, ultrasound wave detector array assembly 22 detects (the beam of) scattered ultrasound waves 32 according to a sinusoidal pulse, with a pulse duration in a range of between about 1 microsecond (μs) and about 1 millisecond (ms). The lower limit of the pulse duration depends upon the frequency of the detected (beam of) scattered ultrasound waves 32, while the upper limit of the pulse duration depends upon the maximum velocity of flowing fluid 12.

For functioning (operating) as described hereinabove, ultrasound wave detector array assembly 22 includes, for example, a linear array of, preferably, at least six, and more preferably, at least thirty, separated, linearly closely spaced apart, and positioned along the same axis (i.e., the y-axis), simultaneously and synchronously operative, transducer type ultrasound wave detectors/receivers. Each such transducer type ultrasound wave detector/receiver is constructed, for example, from a piezoelectric material (e.g., a piezoelectric ceramic based composite material, such as a PZT (lead zirconate titanate) ceramic based composite material), or other appropriate material exhibiting vibrating type of transducer properties, characteristics, and behavior, suitable for detecting/receiving a pulsed beam of scattered ultrasound waves 32. Each such transducer type ultrasound wave detector/receiver converts a pulsed beam of scattered ultrasound waves 32 detected by the piezoelectric material, into an electric voltage, which is subsequently sent, via electrodes, from the piezoelectric material, and therefore, from ultrasound wave detector array assembly 22, to a data acquisition device, for example, data acquisition assembly 24.

For the purpose of decreasing the difference in impedance existing between the outer surface of the detecting/receiving region or zone of ultrasound wave detector array assembly 22 and the outside surface of wall 16b of passage 14, and therefore, for increasing the strength or intensity of the (pulsed beam of) scattered ultrasound waves 32 which exit through wall 16b of passage 14, optionally, a coupling material or substance (not shown in FIG. 2), such as a petroleum based gel or cream, or water, is placed or applied between the outer surface of the detecting/receiving region or zone of ultrasound wave detector array assembly 22 and the outside surface of wall 16b of passage 14.

Data acquisition assembly 24 is for acquiring data sent from ultrasound wave detector array assembly 22. Such data corresponds to near-field amplitude and phase values of the (pulsed beam of) scattered ultrasound waves 32 which exit through wall 16b of passage 14.

Data acquisition assembly 24 is, preferably, suitable for acquiring near-field amplitude and phase values of the (pulsed beam of) scattered ultrasound waves 32, at various frequency components, sent from the linear array of the at least six, separated, linearly closely spaced apart, and positioned along the y-axis, simultaneously and synchronously operative, transducer type ultrasound wave detectors/receivers of ultrasound wave detector array assembly 22.

For functioning (operating) as described hereinabove, data acquisition assembly 24 includes as main components, for example, PC data acquisition cards, a number of simultaneously and synchronously operative lock-in amplifiers and pre-amplifiers, corresponding to the number of linearly arrayed transducer type ultrasound wave detectors/receivers of ultrasound wave detector array assembly 22, along with one or more appropriately connected and operative multiplexers, according to a heterodyne type scheme of data acquisition.

Alternatively, for functioning (operating) as described hereinabove, data acquisition assembly 24 includes main components which are based on effecting direct analog to digital conversion of the near-field amplitude and phase values of the (pulsed beam of) scattered ultrasound waves 32 which exit through wall 16b of passage 14, and sent from ultrasound wave detector array assembly 22.

Central controlling and processing unit 26 is for centrally controlling and processing of functions (operations) and activities, and, associated data and information generated therefrom, of the main components of system 10. More specifically, central controlling and processing unit 26 is for centrally controlling and processing of functions (operations) and activities, and, associated data and information generated (i.e., input, output) therefrom, by main components of system 10, i.e., ultrasound wave transmitter assembly 18, signal generator assembly 20, ultrasound wave detector array assembly 22, and data acquisition assembly 24, according to the preceding illustrative description of the structure and function (operation) of each main component, for implementing the method of the present invention, for ultrasonically determining flow parameters of fluid 12 flowing through a passage, by using far-field analysis.

For functioning (operating) as described hereinabove, central controlling and processing unit 26 is (directly or indirectly) operatively connected to each of the main components of system 10, i.e., ultrasound wave transmitter assembly 18, signal generator assembly 20, ultrasound wave detector array assembly 22, and data acquisition assembly 24. Central controlling and processing unit 26 includes all necessary and appropriate hardware and software suitable for centrally controlling and processing of functions (operations) and activities, and, associated data and information generated therefrom, of the main components of system 10. Such exemplary hardware includes various operatively connected and functioning analog and digital types of electronic controller sub-assemblies and electronic processor sub-assemblies, and components thereof. Such exemplary software includes various operatively connected and functioning written or printed data, in the form of software programs, software routines, software sub-routines, software symbolic languages, software code, software instructions or protocols, software algorithms, or/and combinations thereof.

Central controlling and processing unit 26, preferably, additionally includes a display device, for (real-time or off-line) displaying of the various input or/and output stages of controlling and processing of functions (operations) and activities, and, associated data and information generated therefrom, of the main components of system 10, associated with implementation of the method of the present invention.

In FIG. 2, selected (i.e., not all) operative connections or linkages of electronics, and, data and information communications among main system components, i.e., ultrasound wave transmitter assembly 18, signal generator assembly 20, ultrasound wave detector array assembly 22, and data acquisition assembly 24, and, central controlling and processing unit 26, are generally indicated by (solid) lines drawn between selected (i.e., not all) system components.

### Application of the Generalized System to a Flowing Biological Liquid

Particular exemplary application of the method of the present invention, wherein fluid 12 is a biological liquid (e.g., blood, urine, water), flowing through a vessel, duct, or organ, type of passage 14 of a small scale biological (e.g., human or animal) process, involves special considerations regarding design, construction, and operation, of ultrasound wave transmitter assembly 18 and ultrasound wave detector array assembly 22, of system 10. The main reason for this, is that in such an application, the vessel, duct, or organ, type of passage 14 lies within, and is contained, surrounded, and encompassed, by, a larger body part or/and region thereof, for example, a larger organ, the chest, the head, the pelvis, or a limb. Thus, for performing such an application, the method of the present invention needs to be implemented according to a non-invasive type of medical procedure.

For implementing the method of the present invention according to a non-invasive type of medical procedure, ultrasound wave transmitter assembly 18 and ultrasound wave detector array assembly 22 are not clamped, adhered, or mounted, oppositely facing each other, onto the outside surfaces of walls 16a and 16b, respectively, of the vessel, duct, or organ, type of passage 14. Instead, they are clamped, adhered, or mounted, oppositely facing each other, onto the outside surfaces of the larger body part or/and region thereof, for example, onto the outside surfaces of the larger organ, the chest, the head, the pelvis, or the limb, which contains, surrounds, and encompasses, the vessel, duct, or organ, type of passage 14.

For such an exemplary embodiment of implementing the method of the present invention, ultrasound wave transmitter assembly 18 is designed, constructed, and operative, for transmitting and selectively focusing (a pulsed beam of) ultrasound waves into and through the outside surface (i.e., skin) and internal parts (i.e., tissue, fluids, etc.) of the larger body part or/and region thereof, and into and through wall 16a of the vessel, duct, or organ, type of passage 14, and then into flowing fluid 12 (i.e., biological liquid, e.g., blood, urine, water), in the direction normal or perpendicular to the main or net flow direction of flowing fluid 12, for forming (a pulsed beam of) focused transmitted ultrasound waves, for example, transmitted ultrasound waves 30.

For operating as described hereinabove, ultrasound wave transmitter assembly 18 includes, for example, an ultrasound wave focusing assembly, and a driver or steering assembly. The ultrasound wave focusing assembly is of a structure, for example, having a bowl or bowl-like shape or form, and functions (operates) according to the preceding described selective focusing of ultrasound waves transmitted from ultrasound wave transmitter assembly 18 into fluid 12 (i.e., biological liquid, e.g., blood, urine, water) flowing inside of the vessel, duct, or organ, type of passage 14. The driver or steering assembly is operatively connected to the ultrasound wave focusing assembly, and functions (operates) by selectively steering or driving the ultrasound wave focusing assembly, in a manner suitable for enabling the preceding described transmission and selective focusing of ultrasound waves by ultrasound wave transmitter assembly 18.

Subsequent to the preceding illustratively described transmission and selective focusing of ultrasound waves into flowing fluid 12 (i.e., biological liquid, e.g., blood, urine, water), focused transmitted ultrasound waves 30 propagating through, and scattered by, flowing fluid 12, in the form of scattered ultrasound waves 32, further propagate through wall 16b of the vessel, duct, or organ, type of passage 14, then into and through the internal parts (i.e., tissue, fluids, etc.) and outside surface (i.e., skin) of the larger body part or/and region thereof, and then are detected by ultrasound wave detector array assembly 22.

For such an exemplary embodiment of implementing the method of the present invention, ultrasound wave detector array assembly 22 is, preferably, designed, constructed, and operative, for being complementary to (i.e., simultaneously and synchronously operative with) ultrasound wave transmitter assembly 18. Accordingly, ultrasound wave detector array assembly 22 is designed, constructed, and operative, for detecting (the pulsed beam of) the focused transmitted ultrasound waves 30 which propagate through, and are scattered by flowing fluid 12, as (a pulsed beam of) scattered ultrasound waves 32, which propagate through wall 16b of the vessel, duct, or organ, type of passage 14, then into and through the internal parts (i.e., tissue, fluids, etc.) and outside surface (i.e., skin) of the larger body part or/and region thereof, in the (x-axis) direction normal or perpendicular to the main or net flow (y-axis) direction of flowing fluid 12 (i.e., biological liquid, e.g., blood, urine, water).

The other main components of system 10, i.e., signal generator assembly 20, data acquisition assembly 24, and central controlling and processing unit 26, are appropriately modified or adapted for operating with the preceding described modified structure and function (operation) of ultrasound wave transmitter assembly 18 and ultrasound wave detector array assembly 22, according to the preceding illustratively described transmission and selective focusing of ultrasound waves into fluid 12 (i.e., biological liquid, e.g., blood, urine, water) flowing inside of the vessel, duct, or organ, type of passage 14, and subsequent detection of scattered ultrasound waves 32 thereof.

An exemplary specific embodiment of a system which is suitable, or is readily modified or adapted, for implementing the method of the present invention, and which includes the hereinabove illustratively described main components of system 10 (FIG. 2), is disclosed in the present inventors' teachings [20, 21] of their Near-field/Far-field transformation technique for evaluating and analyzing velocity and vorticity fields in spatial and temporal domains of a (laminar [20] or turbulent [21]) fluid flowing through a passage.

In a non-limiting manner, as stated hereinabove, the method of the present invention is generally implementable by using various different types of equipment and hardware, and associated software, which are known for ultrasonically determining flow parameters of a flowing fluid, and is particularly implementable by using ‘clamp-on’ types of equipment and hardware, and associated software, which include an ultrasound wave transmitter and an ultrasound wave detector array, clamped on, in an oppositely facing configuration, to outer walls of a passage through which the fluid flows, and which operate by transmitting and detecting, respectively, ultrasound waves that propagate normal or perpendicular to the main or net flow direction of the flowing fluid, and are scattered by the flowing fluid.

### Details of Steps of the Method for Determining Flow Parameters of a Flowing Fluid by Using Far-field Analysis

Immediately following is detailed illustrative description of the steps of the method of the present invention (FIG. 1), which is implementable by using the hereinabove illustratively described exemplary generalized preferred embodiment of a clamp-on type of system, i.e., system 10 (FIG. 2), for ultrasonically determining flow parameters of a flowing fluid by using far-field analysis.

### Acquiring Near-Field Amplitude and Phase Change Values of Ultrasound Waves Transmitted Into, Propagating Through, and Scattered by, the Flowing Fluid

In Step (a) of the method for ultrasonically determining flow parameters of a fluid flowing through a passage, by using far-field analysis, of the present invention, there is acquiring near-field amplitude and phase change values of ultrasound waves transmitted into, propagating through, and scattered by, the flowing fluid.

Accordingly, in Step (a), there is acquiring near-field amplitude and phase change values of ultrasound waves transmitted into, propagating through, and scattered by, fluid 12 flowing through passage 14. The near-field amplitude and phase change values are acquired for a measuring plane defined by, and including, a same plane of a transmitting region or zone of an ultrasound wave transmitter device and a detecting region or zone of an ultrasound wave detector array device, wherein the direction of the transmitted ultrasound waves is normal or perpendicular to the main or net longitudinal direction of flow of flowing fluid 12.

As is well known in the art of the present invention, the ‘near-field’ region or zone is characterized and defined by the following condition or relation (1):

b_{NF}≦d²/2λ, (1)

where the parameter b_{NF} is the ‘near-field’ distance extending or spanning from (i) the position or location of a scatterer located within the scattering region or zone of a flowing fluid subjected to, and scattering, ultrasound waves transmitted into, and propagating through, the flowing fluid by an ultrasound wave transmitter device, until (ii) the position or location of the detecting region or zone of an ultrasound wave detector array device detecting the scattered ultrasound waves; the parameter d is the smaller length of either (i) the length of the transmitting region or zone of the ultrasound wave transmitter device or (ii) the length of the detecting region or zone of the ultrasound wave detector array device; and the parameter λ is the wavelength of the transmitted or scattered ultrasound waves.

The parameter, ‘near-field’ distance, b_{NF}, is evaluated for a measuring plane defined by, and including, a same plane of the transmitting region or zone of the ultrasound wave transmitter device and the detecting region or zone of the ultrasound wave detector array device, wherein the direction of the transmitted ultrasound waves is normal or perpendicular to the main or net longitudinal direction of flow of the flowing fluid. In general, for a flowing fluid subjected to, and scattering, transmitted ultrasound waves, each of the parameters, b_{NF}, d, and λ, has a range of values, depending upon the design, construction, and operation, of a particular system, and depending upon the specific properties, characteristics, and behavior, of a particular application, of ultrasonically determining flow parameters of the flowing fluid.

With reference to FIG. 2, the scattering region or zone of flowing fluid 12 generally corresponds to the volumetric region or zone of flowing fluid 12 inside of passage 14 which is subjected to transmitted ultrasound waves 30. As is well known in the art of the present invention, the term ‘scatterer’, for example, scatterer 40 (in FIG. 2, generally indicated by the ‘X’ drawn in flowing fluid 12), generally refers to essentially any feature or characteristic of, or within, flowing fluid 12, which scatters transmitted ultrasonic waves 30 propagating through flowing fluid 12, thereby causing a change in the velocity (speed), as well as the energy, of transmitted ultrasonic waves 30, compared to the velocity (speed), and the energy, of transmitted ultrasonic waves 30 propagating through flowing fluid 12 which are not scattered by the feature or characteristic.

Scatterer 40 is, in general, a feature or characteristic being an internally existing, or/and externally caused, flow fluctuation arising due to a (e.g., velocity, pressure, thermal, concentration, or/and density) inhomogeneity or gradient, or/and turbulence, moving in flowing fluid 12, within the scattering region or zone of flowing fluid 12, which scatters transmitted ultrasonic waves 30 propagating through flowing fluid 12, thereby causing a change in the velocity (speed), and the energy, of transmitted ultrasonic waves 30, compared to the velocity (speed), and the energy, of transmitted ultrasonic waves 30 propagating through flowing fluid 12 which are not scattered by scatterer 40.

Alternatively, or additionally, scatterer 40 is, in general, a feature or characteristic being an internally existing, or/and externally provided (seeded), substance (e.g., particle, droplet, or/and gas bubble) moving in flowing fluid 12, within the scattering region or zone of flowing fluid 12, which scatters transmitted ultrasonic waves 30 propagating through flowing fluid 12, thereby causing a change in the velocity (speed), and the energy, of transmitted ultrasonic waves 30, compared to the velocity (speed), and the energy, of transmitted ultrasonic waves 30 propagating through flowing fluid 12 which are not scattered by scatterer 40.

Accordingly, with reference to FIG. 2, the near-field region or zone is characterized and defined by b_{NF} (in FIG. 2, for example, b_{NF(x)}, indicated by the double-headed arrow) being the near-field distance extending or spanning from (i) the position or location of a scatterer, for example, scatterer 40, located within the scattering region or zone of flowing fluid 12 subjected to, and scattering, ultrasound waves transmitted into, and propagating through, flowing fluid 12 by ultrasound wave transmitter assembly 18, until (ii) the position or location of the detecting region or zone of ultrasound wave detector array assembly 22 detecting scattered ultrasound waves 32; the parameter d is the smaller length of either (i) the length (in FIG. 2, indicated by the length extending along the y-axis direction and between the two d_{T} arrowed reference symbols) of the transmitting region or zone of ultrasound wave transmitter assembly 18 or (ii) the length (in FIG. 2, indicated by the length extending along the y-axis direction and between the two d_{D} arrowed reference symbols) of the detecting region or zone of ultrasound wave detector array assembly 22; and the parameter λ is the wavelength of the transmitted or scattered ultrasound waves 30 or 32, respectively.

The parameter, ‘near-field’ distance, b_{NF(x)}, is evaluated for a measuring plane defined by, and including, a same plane of the transmitting region or zone of ultrasound wave transmitter assembly 18 and the detecting region or zone of ultrasound wave detector array assembly 22, wherein the (x-axis) direction of transmitted ultrasound waves 30 is normal or perpendicular to the main or net (y-axis) longitudinal direction of flowing fluid 12. In general, for flowing fluid 12 subjected to, and scattering, transmitted ultrasound waves 30, each of the parameters, b_{NF}, d, and λ, has a range of values, depending upon the design, construction, and operation, of a particular system, for example, system 10, and depending upon the specific properties, characteristics, and behavior, of a particular application, of implementing the method of the present invention.

Thus, in Step (a), there is using the hereinabove illustratively described exemplary generalized preferred embodiment of a clamp-on type of system, i.e., system 10 (FIG. 2), for acquiring near-field amplitude and phase change values of (a beam of) transmitted ultrasound waves 30 propagating through, and scattered by, flowing fluid 12, in the form of (a beam of) scattered ultrasound waves 32, which exit through wall 16b of passage 14, and are detected by ultrasound wave detector array assembly 22. Data acquisition assembly 24 acquires the near-field amplitude and phase values of the (pulsed beam of) scattered ultrasound waves 32, at various frequency components, sent from the linear array of the at least six, separated, linearly closely spaced apart, and positioned along the y-axis, simultaneously and synchronously operative, transducer type ultrasound wave detectors/receivers of ultrasound wave detector array assembly 22. The acquired near-field amplitude and phase values of the (pulsed beam of) scattered ultrasound waves 32 are sent from data acquisition assembly 24 to central controlling and processing unit 26, for further processing and analysis, in accordance with the method of the present invention.

In Step (a), acquiring of the near-field amplitude and phase values of the (pulsed beam of) scattered ultrasound waves 32, takes place during pre-determined or/and post-determined time intervals, depending upon several factors, such as the design, construction, and operation, of system 10, and the specific properties, characteristics, and behavior, of the particular application involving fluid 12 flowing inside and through passage 14.

Determining a Far-Field Scattering Amplitude Distribution, as a Two-Dimensional Function of Scattering Angle and Doppler Frequency Shift, from the Acquired Near-Field Amplitude and Phase Change Values.

In Step (b) of the method, of the present invention, there is determining a far-field scattering amplitude distribution, as a two-dimensional function of scattering angle and Doppler frequency shift, from the acquired near-field amplitude and phase change values.

Accordingly, in Step (b), there is determining a far-field scattering amplitude distribution, herein, referred to as A(θ, Δf), as a two-dimensional function of scattering angle, herein, referred to as θ, and Doppler frequency shift, herein, referred to as Δf, from the near-field amplitude and phase change values acquired in Step (a). Consistent with Step (a), in Step (b), the far-field scattering amplitude distribution, A(θ, Δf), is determined for a measuring plane defined by, and including, a same plane of a transmitting region or zone of an ultrasound wave transmitter device and a detecting region or zone of an ultrasound wave detector array device, wherein the direction of the transmitted ultrasound waves is normal or perpendicular to the main or net longitudinal direction of flow of flowing fluid 12.

As is well known in the art of the present invention, consistent with condition or relation (1) defining the ‘near-field’ region or zone, the ‘far-field’ region or zone is characterized and defined by the following condition or relation (2):

b_{FF}>>d²/2λ, (2)

where the parameter b_{FF} is the ‘far-field’ distance extending or spanning from (i) the position or location of a scatterer located within the scattering region or zone of a flowing fluid subjected to, and scattering, ultrasound waves transmitted into, and propagating through, the flowing fluid by an ultrasound wave transmitter device, until (ii) a position or location located ‘at or beyond’ the detecting region or zone of an ultrasound wave detector array device detecting the scattered ultrasound waves; and where the parameters, d, and λ, are as defined hereinabove, in Step (a). Consistent with Step (a), in Step (b), the parameter, ‘far-field’ distance, b_{FF}, is evaluated for a measuring plane defined by, and including, a same plane of the transmitting region or zone of the ultrasound wave transmitter device and the detecting region or zone of the ultrasound wave detector array device, wherein the direction of the transmitted ultrasound waves is normal or perpendicular to the main or net longitudinal direction of flow of the flowing fluid.

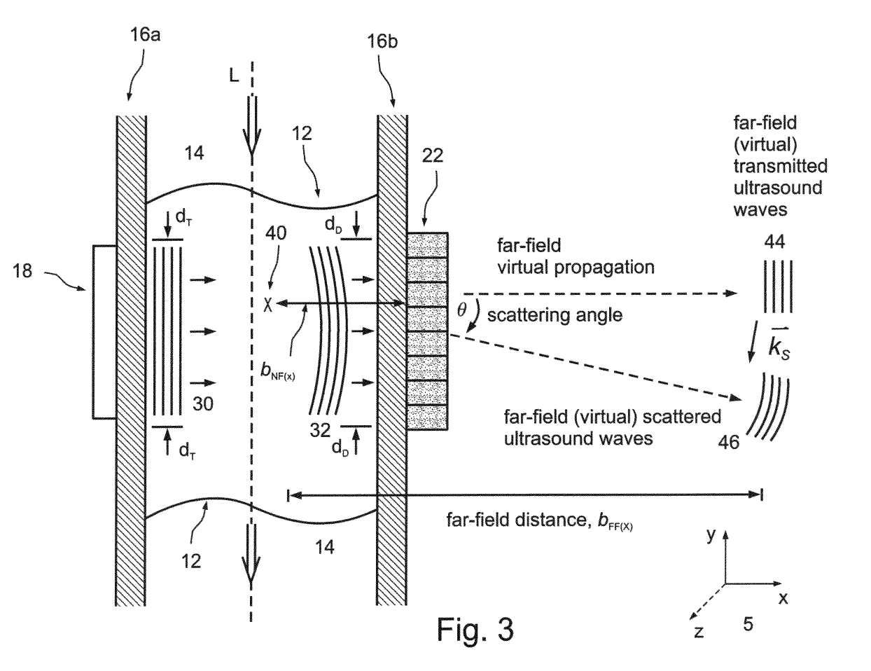

Reference is made to FIG. 3, a schematic diagram illustrating an exemplary embodiment of the main characteristics and parameters of far-field ‘virtual propagation’ of transmitted ultrasound waves 30 and scattered ultrasound waves 32, with respect to system 10 illustrated in FIG. 2, as relating to determining a far-field scattering amplitude distribution, A(θ, Δf), as a two-dimensional function of scattering angle, θ, and Doppler frequency shift, Δf, from near-field amplitude and phase change values of transmitted ultrasound waves 30. In FIGS. 2 and 3, same reference notation and terminology (i.e., numbers, letters, or/and symbols), refer to same components, elements, and parameters, and, in FIG. 3, reference xyz coordinate axis system 5 is shown for indicating x, y, and z, directions relative to the components, elements, and parameters, drawn therein.

Accordingly, with reference to FIG. 3, the far-field region or zone is characterized and defined by b_{FF} (in FIG. 3, for example, b_{FF(x)}, indicated by the double-headed arrow) being the far-field distance extending or spanning from (i) the position or location of a scatterer, for example, scatterer 40, located within the scattering region or zone of flowing fluid 12 subjected to, and scattering, ultrasound waves transmitted into, and propagating through, flowing fluid 12 by ultrasound wave transmitter assembly 18, until (ii) a position or location located ‘at or beyond’ the detecting region or zone of ultrasound wave detector array assembly 22 detecting scattered ultrasound waves 32; and where the parameters, d, and λ, are as defined hereinabove, in Step (a). The parameter, ‘far-field’ distance, b_{FF(x)}, is evaluated for a measuring plane defined by, and including, a same plane of the transmitting region or zone of ultrasound wave transmitter assembly 18 and the detecting region or zone of ultrasound wave detector array assembly 22, wherein the (x-axis) direction of transmitted ultrasound waves 30 is normal or perpendicular to the main or net (y-axis) longitudinal direction of flowing fluid 12.

As shown in FIG. 3, such far-field ‘virtual propagation’ of the ultrasound waves transmitted into, propagating through, and scattered by, the flowing fluid, i.e., near-field (actual) transmitted ultrasound waves 30, and near-field (actual) scattered ultrasound waves 32 thereof, is represented by far-field (virtual) transmitted ultrasound waves 44, and far-field (virtual) scattered ultrasound waves 46, respectively, wherein each beam of ultrasound waves (virtually) propagates in the direction of a far-field (virtual) position or location located at the far-field (virtual) distance, b_{FF(x)}, at or beyond the detecting region or zone of ultrasound wave detector array assembly 22 detecting scattered ultrasound waves 32.

In FIG. 3, the angle by which transmitted ultrasound waves 30 is scattered, thereby forming scattered ultrasound waves 32 thereof, is represented by θ, corresponding to the scattering angle of transmitted ultrasound waves 30. The scattering angle, θ, of transmitted ultrasound waves 30, also corresponds to the angle between the far-field (virtual) propagating path of far-field (virtual) transmitted ultrasound waves 44 and the far-field (virtual) propagating path of far-field (virtual) scattered ultrasound waves 46, in the direction of a far-field (virtual) position or location located at a far-field (virtual) distance, b_{FF(x)}. Additionally, in FIG. 3, the scattering wave vector associated with such ultrasound wave scattering, and such far-field (virtual) propagation thereof, is represented by \vec{k}ₛ.

Such ultrasound wave scattering, and far-field (virtual) propagation thereof, as depicted in FIG. 3, corresponds, in essence, to propagation of transmitted ultrasound waves 30, and scattered ultrasound waves 32 formed therefrom, ‘virtually’ continuing beyond, and outside of, the volumetric region or zone of fluid 12 flowing inside of passage 14, in the form of far-field (virtual) transmitted ultrasound waves 44 and far-field (virtual) scattered ultrasound waves 46, respectively, as if transmitted ultrasound waves 30, and scattered ultrasound waves 32 formed therefrom, were still propagating within, and inside of, the volumetric region or zone of fluid 12 flowing inside of passage 14. This corresponds to the conceptual basis of the present inventors' initially developed Near-field/Far-field transformation technique [20, 21].

Thus, in view of the hereinabove definitions, and illustrative descriptions thereof, of the near-field and far-field ultrasound wave transmission and scattering phenomena, in Step (b), there is determining a far-field scattering amplitude distribution, A(θ, Δf), as a two-dimensional function of the scattering angle, θ, and Doppler frequency shift, Δf, from the near-field amplitude and phase change values acquired in preceding Step (a).

Step (b) includes utilizing the present inventors' initially developed Near-field/Far-field transformation technique [20, 21]. As previously described hereinabove, in the Background section, the Near-field/Far-field transformation technique basically corresponds to ‘transforming’, ‘projecting’, ‘extrapolating’, or ‘mapping’, data and information (i.e., properties, characteristics, and behavior) of ultrasound waves which are transmitted, scattered, and detected, in the near-field region or zone, of a fluid flowing in and through a passage, from the near-field region or zone to the far-field region or zone, of the flowing fluid, and using the transformed, projected, extrapolated, or mapped, data and information for evaluating and analyzing flow parameters (particularly, peak velocity, velocity distribution, and flow rate) of the fluid flowing in and through the passage.

Accordingly, for implementing the method of the present invention, Step (b) includes transforming, projecting, extrapolating, or mapping, data and information (i.e., properties, characteristics, and behavior), in particular, in the form of acquired near-field amplitude and phase change values, of ultrasound waves which are transmitted, scattered, and detected, in the near-field region or zone (as defined by condition or relation (1) and illustratively described hereinabove, with reference to FIGS. 2 and 3), of fluid 12 flowing in and through passage 14, from the near-field region or zone to the far-field region or zone (as defined by condition or relation (2) and illustratively described hereinabove, with reference to FIG. 3), of flowing fluid 12, and using the transformed, projected, extrapolated, or mapped, data and information (i.e., the acquired near-field amplitude and phase change values) for determining a far-field scattering amplitude distribution, A(θ, Δf), as a two-dimensional function of the scattering angle, θ, and Doppler frequency shift, Δf.

Step (b) is performed according to either of two alternative cases, i.e., a first case, based on using the acquired near-field amplitude and phase change values expressed in terms of time series in the time domain, or, alternatively, a second case, based on using the acquired near-field amplitude and phase change values expressed in terms of frequency components in the frequency domain, where the first case and the second case differ according to the order of using a Fourier transform procedure. In each of the first and second cases, the time series of the acquired near-field amplitude and phase change values corresponds to a mode of pulses of the detected scattered ultrasound waves (scattered ultrasound waves 32), or a mode of direct analog to digital conversion of the detected scattered ultrasound waves (scattered ultrasound waves 32).

By using the present inventors' initially developed Near-field/Far-field transformation technique [20, 21], Step (b) of the method of the present, includes constructing a far-field scattering wave function in terms of a near-field scattering wave function (according to the first case, for any given instant of time, i.e., in terms of the time domain, or, alternatively, according to the second case, for any given frequency component, i.e., in terms of the frequency domain) from the acquired near-field amplitude and phase change values (from Step (a)). Construction of the far-field scattering wave function in terms of a near-field scattering wave function is based on application of a mathematical description of the Huygens' Principle (conventionally directed to optics and propagation of electromagnetic radiation) to the near-field measurements (i.e., the acquired near-field amplitude and phase change values (from Step (a)) of the ultrasound waves scattered by fluid 12 flowing through passage 14, and is derived from the Rayleigh-Sommerfeld integral in accordance with the following equation (3):

$${Ψ ( {r}_{f} , y )}_{scat}^{ff} = \int \frac{{k}_{0} {i}^{3 / 2} d {y}^{′}}{\sqrt{2 π {k}_{0} ( {r}_{f} - {r}_{d} )}} {e}^{i [ {{k}_{0} ( y - {y}^{′} )}^{2} ] / [ 2 ( {r}_{f} - {r}_{d} ) ]} {Ψ ( {r}_{d} , {y}^{′} )}_{scat}^{d} \qquad ( 3 )$$

where the terms in equation (3) are defined as follows:

r_{d} is the ‘near-field’ radial distance, extending or spanning from a radial center position or location located within passage 14, until the detecting region or zone of ultrasound wave detector array assembly 22 detecting scattered ultrasound waves 32, in the near-field region or zone (as defined by condition or relation (1) and illustratively described hereinabove, with reference to FIGS. 2 and 3) of flowing fluid 12;

r_{f} is the ‘far-field’ radial distance extending or spanning from a radial center position or location within passage 14, until a far-field (virtual) position or location located at the far-field (virtual) distance, b_{FF}, at or beyond the detecting region or zone of ultrasound wave detector array assembly 22 detecting scattered ultrasound waves 32, in the far-field region or zone (as defined by condition or relation (2) and illustratively described hereinabove, with reference to FIG. 3), of flowing fluid 12;

Ψ(r_{d},y′)_{scat}^{d} is the ‘near-field’ scattering wave function in the near-field region or zone (as defined by condition or relation (1) and illustratively described hereinabove, with reference to FIGS. 2 and 3) of flowing fluid 12;

Ψ(r_{f}, y)_{scat}^{ff} is the ‘far-field’ scattering wave function at a far-field (virtual) position or location located at the far-field (virtual) distance, b_{FF}, at or beyond the detecting region or zone of ultrasound wave detector array assembly 22 detecting scattered ultrasound waves 32, in the far-field region or zone (as defined by condition or relation (2) and illustratively described hereinabove, with reference to FIG. 3), of flowing fluid 12;

y is the (y-axis) coordinate along the detecting region or zone of the linear array of the at least six, separated, linearly closely spaced apart, and positioned along the y-axis, simultaneously and synchronously operative, transducer type ultrasound wave detectors/receivers of ultrasound wave detector array assembly 22;

y′ is the integration variable, corresponding to variable y;

k₀ is the wavenumber of transmitted ultrasound waves 30, defined as k₀=2π/λ, where λ is the wavelength of transmitted ultrasound waves 30; and

π is the well known symbol for ‘pi’, the (transcendental) mathematical (constant) number, 3.14 . . . .

The far-field scattering wave function, Ψ(r_{f}, y)_{scatt}^{ff} as defined by equation (3), can be expressed as a function of the scattering angle, θ, as provided by the following equation (4):

\tilde{Ψ}(θ)=Ψ(r_{f},y)_{scat}^{ff}|_{θ=arctan(y/r}_{_{f}}), (4)

where the right hand side of equation (4) is a function of the scattering angle, θ, which is evaluated from the arctan of (y/r_{f}).

### First Case: Using the Acquired Near-Field Amplitude and Phase Change Values Expressed in Terms of the Time Domain

The first case is based on, and includes, using the acquired near-field amplitude and phase change values (from Step (a)) expressed in terms of time series in the time domain, for constructing a far-field scattering wave function in terms of the time domain, which is then transformed (via using a Fourier transform procedure) from the time domain to the frequency domain. Accordingly, in the first case, by subjecting the constructed far-field scattering wave function, equation (4), to a Fourier transform procedure, herein, referred to as F_{(Δf)}, there is separating the constructed far-field scattering wave function into different frequency components.

Performing the first case of Step (b), results in the following equation (5):

\tilde{Ψ}(θ,Δf)=F_{(Δf)}{\tilde{Ψ}(θ)₍ₜ₎} (5)

where the bracketed right hand side of equation (5) corresponds to the far-field scattering wave function, expressed as a function of the scattering angle, θ, as defined by equation (4), in terms of the time domain (t). Equation (5) corresponds to the far-field scattering wave function as a two-dimensional function of scattering angle, θ, and Doppler frequency shift, Δf.

### Second Case: Using the Acquired Near-Field Amplitude and Phase Change Values Expressed in Terms of the Frequency Domain

The second case is based on, and includes, transforming (via using a Fourier transform procedure) the acquired near-field amplitude and phase change values (from Step (a)) expressed in terms of time series in the time domain, from the time domain to the frequency domain, and using the acquired near-field amplitude and phase change values expressed in terms of the frequency domain, for constructing the far-field scattering wave function in terms of the frequency domain. Accordingly, in the second case, by subjecting the time series of the acquired near-field amplitude and phase change values to a Fourier transform procedure, there is separating the acquired near-field amplitude and phase change values into different frequency components, which are then used, via equation (3), for writing the far-field scattering wave function.

Performing the second case of Step (b), results in the following equation (6):

\tilde{Ψ}(θ,Δf)=\tilde{Ψ}(θ)_{(Δf)} (6)

where the right hand side of equation (6) corresponds to the far-field scattering wave function, expressed as a function of the scattering angle, θ, as defined by equation (4), in terms of the frequency domain (i.e., via the Doppler frequency shift, Δf). Equation (6) corresponds to the far-field scattering wave function as a two-dimensional function of scattering angle, θ, and Doppler frequency shift, Δf.
Far-Field Scattering Amplitude Distribution, as a Two-Dimensional Function of the Scattering angle, θ, and Doppler frequency shift, Δf

Following performing each of the first case, or, alternatively, the second case, Step (b) is completed by taking normal or absolute values of the far-field scattering wave function as a two-dimensional function of scattering angle, θ, and Doppler frequency shift, Δf, using either equation (5) of the first case, or, alternatively, using equation (6) of the second case, for obtaining the far-field scattering amplitude distribution, A(θ, Δf), as a two-dimensional function of the scattering angle, θ, and Doppler frequency shift, Δf, as provided by the following equation (7a):

A(θ,Δf)=|\tilde{Ψ}(θ,Δf)| (7a)

Equation (7a) corresponds to the far-field scattering amplitude distribution, as a two-dimensional function of the scattering angle, θ, and Doppler frequency shift, Δf.

Optionally, Equation (7a) can be replaced by the following Equation (7b):

A(θ,Δf)=|\tilde{Ψ}(θ,Δf)|−|\tilde{Ψ}(−θ,Δf)| (7b)

which corresponds to a procedure of introducing a filter for reducing background scattering and background electronic noise from the result provided by equation (7a). This procedure is recommended in unidirectional flow to improve the overall accuracy of the flow rate measurement.

As previously illustratively described hereinabove, subsequent to transmission of ultrasound waves into flowing fluid 12, transmitted ultrasound waves 30 formed therefrom, propagate through flowing fluid 12 in the (x-axis) direction normal or perpendicular to the main or net flow (y-axis) direction of flowing fluid 12, and therefore, propagate through flowing fluid 12 in the direction normal or perpendicular to longitudinal axis L of passage 14. Scattering of transmitted ultrasound waves 30 by flowing fluid 12, in the form of scattered ultrasound waves 32, occurs within flowing fluid 12 which has a velocity component along the same (x-axis) direction of (the beam of) transmitted ultrasound waves 30 (i.e., normal or perpendicular to longitudinal axis L of passage 14), and exit through wall 16b of passage 14, and are detected by ultrasound wave detector array assembly 22.

By performing Step (b) of the method of the present, the present inventors' initially developed ‘Near-field/Far-field’ transformation technique [20, 21] is further extended, and used herein, in a novel and inventive way, as part of implementing the present invention, for constructing the far-field scattering wave function as a two-dimensional function of scattering angle, θ, and Doppler frequency shift, Δf, (i.e., as provided by equation (5), according to the hereinabove described first case, for any given instant of time, in terms of the time domain, or, alternatively, as provided by equation (6), according to the hereinabove described second case, for any given frequency component, in terms of the frequency domain) from the near-field amplitude and phase change values acquired in Step (a). Step (b) ultimately results in determination of the far-field scattering amplitude distribution, A(θ, Δf), as a two-dimensional function of the scattering angle, θ, and Doppler frequency shift, Δf, as provided by equation (7).

The far-field scattering amplitude distribution, A(θ, Δf), determined in Step (b), is used for performing Step (c), as illustratively described hereinbelow, for determining flow parameters (particularly, peak velocity, velocity distribution, and flow rate) of flowing fluid 12, in the (y-axis) direction normal or perpendicular to the (x-axis) direction of transmitted ultrasound waves 30, i.e., in the (y-axis) longitudinal direction of flow of flowing fluid 12, in a measuring plane defined by, and including, the transmitting region or zone of ultrasound wave transmitter assembly 18 and the detecting region or zone of ultrasound wave detector array assembly 22, which is of significant current interest in a wide variety of different fluid flow applications.

In addition to the present inventors' justification for initially developing, and applying, the Near-field/Far-field transformation technique [20, 21], a main limitation associated with prior art techniques for detecting scattering of ultrasound waves which are transmitted and scattered in the near-field region or zone of a fluid flowing in and through a passage (e.g., scattered ultrasound waves 32 of fluid 12 flowing in and through passage 14), is that the amplitude of the scattered ultrasound waves (i.e., scattered ultrasound waves 32) attenuates (i.e., decreases) as a function of increasing scattering angle, O. Such amplitude is typically hidden or ‘buried’ within the amplitude of (the beam of) transmitted ultrasound waves 30 propagating through, and scattered by, flowing fluid 12.

Only in the far-field region or zone (as defined by condition or relation (2) and illustratively described hereinabove, with reference to FIG. 3), of flowing fluid 12, is the amplitude of the far-field (virtual) scattered ultrasound waves 46, (as defined by the far-field scattering amplitude distribution, A(θ, Δf), according to equation (7)), as a function of increasing scattering angle, θ, separable from the amplitude of the far-field (virtual) transmitted ultrasound waves 44, where such ultrasound waves (virtually) propagate in the direction of a far-field (virtual) position or location located at a far-field (virtual) distance, b_{FF}, at or beyond the detecting region or zone of ultrasound wave detector array assembly 22 detecting scattered ultrasound waves 32.

Determining the Flow Parameters of the Flowing Fluid, from the Far-Field Scattering Amplitude Distribution.

In Step (c) of the method, of the present invention, there is determining flow parameters of the flowing fluid, from the scattering amplitude distribution.

Accordingly, in Step (c), there is determining flow parameters (particularly, peak velocity, velocity distribution, and flow rate) of flowing fluid 12, from the scattering amplitude distribution, A(θ, Δf), determined in Step (b).

Peak Velocity, vₚₑₐₖ, of the Flowing Fluid

The peak velocity of flowing fluid 12 is the value of the velocity which corresponds to a ‘peak’ in the distribution function of the velocity component of flowing fluid 12 which is normal or perpendicular to the (x-axis) direction of (the beam of) transmitted ultrasound waves 30. For specific properties, characteristics, and behavior, of fluid 12 flowing through and along passage 14, there may exist more than a single peak velocity of flowing fluid 12.

Peak velocity, herein, referred to as vₚₑₐₖ, of flowing fluid 12, is determined by using the following equation (8) of the Doppler frequency shift, Δf:

Δf=\vec{v}·\vec{k}_{S}/2π, (8)

where the terms in equation (8) are defined as follows:

Δf is the Doppler frequency shift that corresponds to each examined point (per scattering angle, θ) in the far-field scattering amplitude distribution, A(θ, Δf) (determined in Step (b) and defined by equation (7));

\vec{v} is the mean or net (advection) moving velocity of the various scatterers (for example, scatterer 40 (FIGS. 2, 3), as illustratively described hereinabove in Step (a), with reference to FIG. 2) moving in flowing fluid 12, in the (y-axis) longitudinal direction of passage 14, within the scattering region or zone of flowing fluid 12, which scatter transmitted ultrasonic waves 30 propagating through flowing fluid 12. [A given scatterer is an internally existing, or/and externally caused, flow fluctuation arising due to a (e.g., velocity, pressure, thermal, concentration, or/and density) inhomogeneity or gradient, or/and turbulence, moving in flowing fluid 12, or/and an internally existing, or/and externally provided (seeded), substance (e.g., particle, droplet, or/and gas bubble) moving in flowing fluid 12, in the (y-axis) longitudinal direction of passage 14, within the scattering region or zone of flowing fluid 12, which scatters transmitted ultrasonic waves 30 propagating through flowing fluid 12.]; and

\vec{k}_{S} is the scattering wave vector, as illustratively described hereinabove in Step (b), with reference to FIG. 3, associated with scattering of transmitted ultrasound waves 30, and far-field (virtual) propagation thereof.

With reference to FIG. 3, for small scattering angles, i.e., small θ, scattering of (the beam of) transmitted ultrasound waves 30, in the form of (the beam of) scattered ultrasound waves 32, is well detectable. At a far-field (virtual) position or location located at a far-field (virtual) distance, b_{FF(x)}, at which far-field (virtual) propagation takes place, in the form of far-field (virtual) transmitted ultrasound waves 44 and far-field (virtual) scattered ultrasound waves 46, respectively, the scattering wave vector, \vec{k}_{S}, is approximately transverse (normal or perpendicular) to the (x-axis) direction of (the beam of) transmitted ultrasound waves 30 (and to the (x-axis) direction of (the beam of) far-field (virtual) transmitted ultrasound waves 44). In the limit of zero scattering angle, i.e., θ=0, to the direction of the scattering wave vector, \vec{k}_{S}, approaches the (y-axis) direction normal or perpendicular to the (x-axis) direction of (the beam of) transmitted ultrasound waves 30 (i.e., in the (y-axis) longitudinal direction of flow of flowing fluid 12, in a measuring plane defined by, and including, the transmitting region or zone of ultrasound wave transmitter assembly 18 and the detecting region or zone of ultrasound wave detector array assembly 22), and, the magnitude of the scattering wave vector, \vec{k}_{S}, depends on the scattering angle, θ, according to the following relation (9):

k_{S}≈k₀θ, (9)

where k₀ is the wavenumber of transmitted ultrasound waves 30, as defined hereinabove in Step (b), in the context of equation (3) defining the ‘far-field’ scattering wave function, Ψ(r_{f},y)_{scat}^{ff}.

Therefore, from equations (8) and (9), it follows that the peak velocity, vₚₑₐₖ, of the velocity component of flowing fluid 12 which is normal or perpendicular to the (x-axis) direction of (the beam of) transmitted ultrasound waves 30 is defined by, and determined from, the following equation (10):

vₚₑₐₖ=(2π/k₀)(∂Δf/∂θ)|_{crest}, (10)

where (∂Δf/∂θ)|_{crest} is the partial derivative of the Doppler frequency shift, Δf, with respect to the scattering angle, θ. The peak velocity, vₚₑₐₖ, provided by equation (10) corresponds to the slope, in terms of the axis of the Doppler frequency shift, Δf, with respect to the axis of the scattering angle, θ, of the ‘best fitting’ line of a crest, or crest-like, shape or form, in a graphical plot of the far-field scattering amplitude distribution, A(θ, Δf), two-dimensional function (i.e., surface), and is empirically determined from flow measurements of flowing fluid 12, for example, by using system 10 for implementing the present invention. The peak velocity, vₚₑₐₖ, provided by equation (10) also corresponds to the direction of a crest, or crest-like, shape or form, which is ‘visually’ observable in a graphical plot of the far-field scattering amplitude distribution, A(θ, Δf), two-dimensional function (surface).

### Velocity Distribution of the Flowing Fluid

The velocity distribution of flowing fluid 12 is determined in terms of a probability distribution function, herein, referred to as P_{d}(v), where v is the velocity component of flowing fluid 12 which is normal or perpendicular to the (x-axis) direction of (the beam of) transmitted ultrasound waves 30.

The velocity distribution of flowing fluid 12 is determined from the preceding described crest, or crest-like, shape or form, visually observed in a graphical plot of the far-field scattering amplitude distribution, A(θ, Δf), two-dimensional function (surface), and used for describing the mathematical form of the peak velocity, vₚₑₐₖ, of flowing fluid 12, as defined by preceding equation (10). In some detection systems, instead of fitting a line as described in the preceding section, it may be preferable to find the peak velocity, vₚₑₐₖ, directly from the peak in the probability distribution function, P_{d}(v), as described in the following.

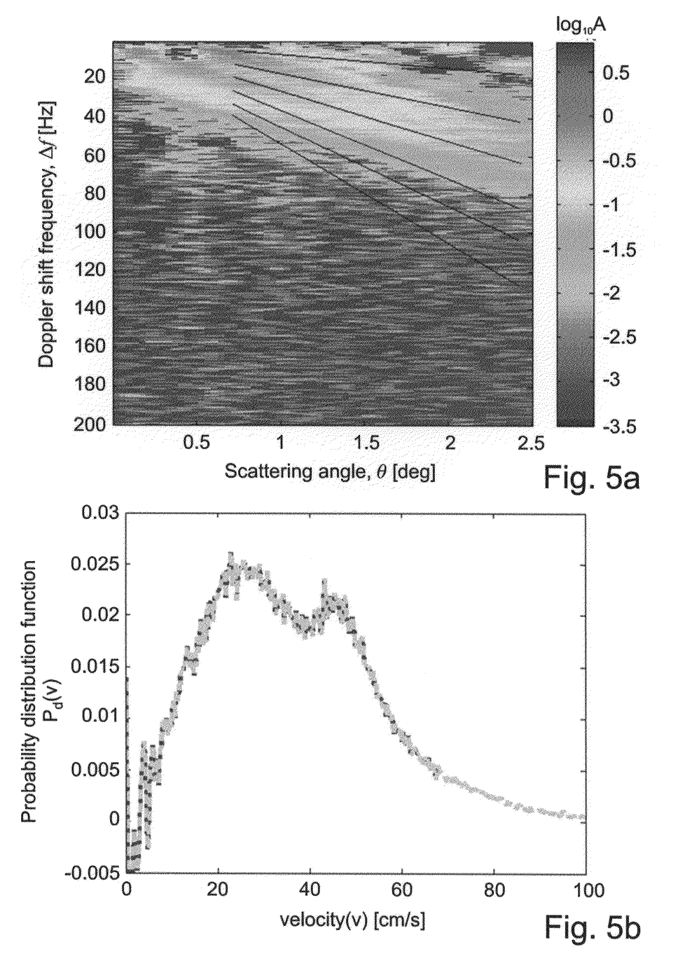

In a graphical representation, the weight of the probability distribution function, P_{d}(v), at a particular velocity, v, is proportional to the average value over a section of line intercepting the origin of axes (θ, Δf) [for example, as shown hereinbelow, in FIG. 5a of Example 2] of a two dimensional function far-field scattering amplitude distribution, A(θ, Δf). The velocity, v, that corresponds to that line is obtained from the tangent of the line in terms of Δf/θ, based on the formula v=[2π·Δf]/[k₀·θ], obtained from rearrangement of preceding equation (8) of the Doppler frequency shift, Δf.

The probability distribution function, P_{d}(v), of the velocity, v, is defined by the following equation (11a):

$${P}_{d} ( v ) = {\int }_{{θ}_{1}}^{{θ}_{2}} d θ A ( θ , \frac{{vk}_{0}}{2 π} θ ) / N \qquad ( 11 a )$$

Practical calculation of the probability distribution function, P_{d}(v), is determined from a nearest neighbor interpolation of a matrix, Aᵢₙₜ, along the lines of different slopes, using the following equation (11b):

$${P}_{d} ( v ) = {\sum }_{{θ}_{i} = {θ}_{1}}^{{θ}_{2}} {A}_{intp} ( {θ}_{i} , \frac{{vk}_{0}}{2 π} {θ}_{i} ) / \tilde{N} \qquad ( 11 b )$$

where Ñ is a normalization factor, θ₁ and θ₂ designate the range of scattering angles that the scattering amplitude is well detected in the system (for example, values used in practice for an ultrasound frequency of 5 MHz are about θ₁=0.5° and θ₂=2.5°), and where k₀ is the wavenumber of transmitted ultrasound waves 30 (FIGS. 2 and 3) as defined hereinabove in Step (b), in the context of equation (3) defining the ‘far-field’ scattering wave function, Ψ(r_{f},y)_{scat}^{ff}.

In addition, since there is remaining background scattering and background noise that should not be included in the probability distribution function, P_{d}(v), of the velocity, it is required to have a condition which truncates the probability distribution function, P_{d}(v), of the velocity at a certain truncation point or maximum velocity. The truncation point or maximum velocity, vₘₐₓ, is the highest value of the velocity, v, where the sum expressed by the following equation (11c):

$${\sum }_{{θ}_{i} = {θ}_{1}}^{{θ}_{2}} {A}_{intp} ( {θ}_{i} , \frac{{vk}_{0}}{2 π} {θ}_{i} ) / {\sum }_{{θ}_{i} = {θ}_{1}}^{{θ}_{2}} 1 \qquad ( 11 c )$$

becomes lower than a threshold value that is determined according to the background scattering and background noise in the specific system. Accordingly, this condition is expressed (with suitable adjustment of the normalization factor, Ñ) by the following equation (11d):

P_{d}(v>vₘₐₓ)=0 (11d)

### Flow Rate, Q, of the Flowing Fluid

The flow rate, herein, referred to as Q, of flowing fluid 12, is determined in terms of (i) the peak velocity, vₚₑₐₖ, of flowing fluid 12, as defined by preceding equation (10); (ii) the probability distribution function, P_{d}(v), of the velocity, as defined and determined by preceding equations (11a, 11b, 11c, and 11d); (iii) the value of the cross-sectional area of passage 14 through which flows fluid 12; and (iv) a statistical geometrical factor, herein, referred to as G{P_{d}}, representing a function of various geometrical characteristics and parameters relating to flowing fluid 12, passage 14, transmission of the ultrasound waves into flowing fluid 12, and measurement of scattered ultrasound waves 32.

More specifically, the statistical geometrical factor, G{P_{d}}, is a function of several geometrical characteristics and parameters, particularly, (1) the geometrical shape, form, and dimensions (such as the diameter), of passage 14 through which flows fluid 12; (2) the extent by which flowing fluid 12 occupies or fills the cross-sectional area of passage 14; (3) the extent by which (the beam of) transmitted ultrasonic waves 30 propagate through the cross-sectional area of passage 14 through which flows fluid 12; (4) the geometrical shape, form, and dimensions, of the transmitting region or zone of ultrasound wave transmitter assembly 18; and (5) the geometrical shape, form, and dimensions, of the detecting region or zone of ultrasound wave detector array assembly 22. Accordingly, the statistical geometrical factor, G{P_{d}}, depends upon the quantity and extent by which fluid 12 flows through passage 14, and depends upon the design, construction, and operation, of a particular system, for example, system 10, used for implementing the method of the present invention.

For example, in the case wherein (the beam of) transmitted ultrasonic waves 30 propagate through the entire cross-sectional area of passage 14 through which flows fluid 12, then, the statistical geometrical factor, G{P_{d}}, corresponds to a coefficient having a constant value. Thus, the flow rate, Q, of flowing fluid 12, is defined by, and determined from, the following equation (12):

Q=G{P_{d}}∫dv′v′P_{d}(v′). (12)

Additionally, for example, in the particular case wherein the thickness (size along the z-axis) of (the beam of) transmitted ultrasonic waves 30 is much less than the diameter of passage 14, then, the statistical geometrical factor, G{P_{d}}, can be approximated by the following equation (13):

$$G \{ {P}_{d} \} = \frac{2 n}{2 n + 1} S , \qquad ( 13 )$$

where S is the cross-sectional area of passage 14, and n is a number having a value of about 7 (according to the model of turbulent flow in a circular pipe, known as “the 7th root law” [26]. More specifically, according to the flow rate flowing fluid 12, n changes from about 6 at the onset of turbulence to about 10 at a Reynolds number on the order of several millions.

Above illustratively described novel and inventive aspects and characteristics, and advantages thereof, of the present invention further become apparent to one ordinarily skilled in the art upon examination of the following example, which is not intended to be limiting. Additionally, each of the various embodiments and aspects of the present invention as delineated herein above and as claimed in the claims section below finds experimental support in the following examples.

### EXAMPLES

Reference is now made to the following examples, which together with the above description, illustrate the invention in a non-limiting fashion.

### Example 1

### Ultrasonically Determining Flow Parameters of a Fluid (Liquid Water)Flowing Through a Passage (Silicon Rubber Pipe)

In Example 1, there was ultrasonically determining flow parameters of a fluid (pure liquid water) flowing through a passage (silicon rubber pipe). The silicon rubber pipe had an inside diameter of 5 millimeters (mm), and a wall thickness of 2 millimeters (mm).

### Experimental Procedure

Example 1 was performed by using the same experimental measuring system which was used, and disclosed, in the present inventors' teachings [20, 21] of their initially developed Near-field/Far-field transformation technique for evaluating and analyzing velocity and vorticity fields in spatial and temporal domains of a laminar or turbulent fluid flowing through a passage. The experimental measuring system was specially adapted and modified for ultrasonically determining flow parameters of a pure liquid water type of fluid flowing through a silicon rubber pipe type of passage, for implementing the method of the present invention (as illustratively described hereinabove, with reference to FIGS. 1-3). Water was used as the acoustic coupling material between the outside surface of the silicon rubber pipe and the transmitter and detector arrays.

The experimental measuring system corresponded to a ‘clamp-on’ type of experimental measuring system, based on equipment and hardware, and associated software, which included an ultrasound wave transmitter and an ultrasound wave detector array device, clamped on, in an oppositely facing configuration, to the outer walls of the silicon rubber pipe through which the pure liquid water flowed, and which operated by transmitting and detecting, respectively, ultrasound waves that propagated normal or perpendicular to the main or net flow direction of the flowing liquid water, and were scattered by the flowing liquid water. Accordingly, the experimental measuring system included the hereinabove illustratively described main components of system 10 (FIG. 2).

In accordance with Step (a) of the method of the present invention, for obtaining near-field measurements of amplitude and phase change values of ultrasound waves transmitted into, propagating through, and scattered by, the flowing water, a highly coherent finite width (100 millimeter (mm)), (pulsating) beam of ultrasound waves (sinusoidal, frequency of 5.5 MHz, and pulse duration of 5 μs) was generated by a transducer type ultrasound wave transmitter (constructed from a composite piezoelectric material), whose transmitting region or zone was 100 millimeter (mm) long, and the ultrasound wave pulses were transmitted into, propagated through, and scattered by, the water flowing inside and through the silicon rubber pipe type of passage.

Amplitude and phase of the scattered ultrasound waves were detected by a linear detector array of 62 separate, closely spaced apart by 1 millimeter (mm), simultaneously and synchronously operative ultrasound wave detectors (facing opposite, across from, and lying in the same plane as, the ultrasound wave transmitter), which were operative with two PC data acquisition cards, 62 lock-in amplifiers, and a corresponding number of pre-amplifiers, according to a heterodyne type scheme of data acquisition. Simultaneous sampling of the ultrasound wave detectors over the linear detector array channels was done 1,800 times per second.

In accordance with Step (b) of the method of the present invention, the far-field scattering amplitude distribution, A(Δ, θf), as a two-dimensional function of scattering angle, θ, and Doppler frequency shift, Δf, as defined hereinabove by equation (7), was determined from the preceding described acquisition of near-field amplitude and phase change values.

In accordance with Step (c) of the method of the present invention, flow parameters of the flowing water were determined from the preceding determined scattering amplitude distribution, A(θ, Δf).

### Experimental Results

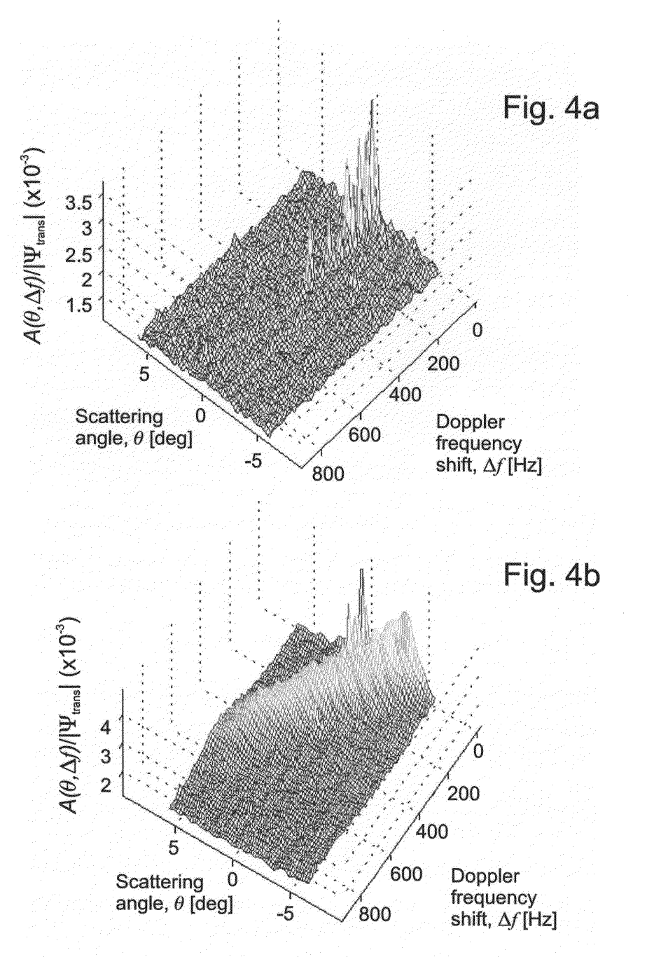

The results obtained for Example 1 are presented in FIGS. 4a and 4b, which are three-dimensional graphical presentations of exemplary results of implementing the method of the present invention, for ultrasonically determining flow parameters of a pure liquid water type of fluid flowing through a silicon rubber pipe type of passage.

### For Non Flow (Background Noise) Conditions

FIG. 4a is a plot of the ‘background noise’ far-field scattering amplitude distribution, A(θ, Δf), ‘normalized’ with respect to the transmitted ultrasound wave amplitude, |Ψₜᵣₐₙₛ|, as a function of the scattering angle, θ, and Doppler frequency shift, Δf, for non-flow conditions, wherein the transmitted ultrasound waves were scattered by the non-flowing (stationary) water in the pipe.

From the results graphically presented in FIG. 4a, it is concluded that, for non-flow conditions, the peak of the normalized far-field scattering amplitude distribution, A(θ, Δf)/|Ψₜᵣₐₙₛ| appearing near the zero value of scattering angle, θ, corresponds to the main signal of the beam of the transmitted ultrasound waves spread around the zero value (i.e., absence) of the Doppler frequency shift, Δf. This angular offset is due to an offset (misalignment) of the direction of the beam of the transmitted ultrasound waves relative to the plane of the linear array of the ultrasound wave detectors clamped onto the pipe. Even though the pipe material causes or induces scattering of the beam of the transmitted ultrasound waves, such relatively insignificant scattering was absent during the Doppler frequency shift analysis, and only the main beam of the transmitted ultrasound waves is of significance.

### For Flow Conditions

FIG. 4b is a plot of the ‘actual’ far-field scattering amplitude distribution, A(θ, Δf), ‘normalized’ with respect to the transmitted ultrasound wave amplitude, |Ψₜᵣₐₙₛ|, as a function of the scattering angle, θ, and Doppler frequency shift, Δf, for flow conditions, wherein the transmitted ultrasound waves were scattered by the water flowing (moving) through the pipe.

From the results graphically presented in FIG. 4b, it is concluded that, for flow conditions, the finger-like pattern of the (Doppler) peak of the normalized far-field scattering amplitude distribution, A(θ, Δf)/|Ψₜᵣₐₙₛ|, corresponds to the ‘actual’ far-field scattering amplitude distribution of the scattered ultrasound waves, as a function of the scattering angle, θ, and Doppler frequency shift, Δf.

The peak velocity, vₚₑₐₖ, being a positive or negative magnitude of the velocity component of the flowing water which is normal or perpendicular to the direction of the beam of the transmitted ultrasound waves was evaluated from equation (10) (as described hereinabove, in Step (c)):

vₚₑₐₖ=(2π/k₀)(∂Δf/∂θ)|_{crest}, (10)

where (∂Δf/∂θ)|_{crest} is the partial derivative of the Doppler frequency shift, Δf, with respect to the scattering angle, θ, corresponding to the slope, in terms of the Doppler frequency shift, Δf, axis, with respect to the scattering angle, θ, axis, of the ‘best fitting’ line of the crest of the finger-like pattern of the (Doppler) peak of the normalized far-field scattering amplitude distribution, A(θ,Δf)/|Ψₜᵣₐₙₛ|, observed in FIG. 4b.

Based on the (operator) ‘known’ or pre-set (and measured) flow rate of the water flowing through the pipe, the (operator) known or pre-set peak velocity, vₚₑₐₖ, of the flowing water was calculated to be 1.7±0.1 meters per second (m/s).

In accordance with Step (c) of the method of the present invention, from equation (10), the ‘ultrasonically’ determined peak velocity, vₚₑₐₖ, of the water flowing through the pipe was evaluated to be 1.5±0.2 meters per second (m/s), with uncertainty due to misalignment of the direction of the beam of the transmitted ultrasound waves relative to the plane of the linear array of the ultrasound wave detectors clamped onto the pipe. The ‘ultrasonically’ determined peak velocity, vₚₑₐₖ, of the flowing water is in relatively good agreement with the (operator) known or pre-set peak velocity, vₚₑₐₖ, of the flowing water, thus providing a good exemplary indication of the accuracy of implementing the method of the present invention.

The velocity distribution of the water flowing through the pipe is determined from the preceding described crest of the finger-like pattern of the (Doppler) peak of the normalized far-field scattering amplitude distribution, A(θ, Δf)/|Ψₜᵣₐₙₛ|, observed in FIG. 4b.

### Example 2

### Ultrasonically Determining Flow Parameters of a Fluid (Liquid Water) Flowing Through a Passage (Painted Steel Pipe)

In Example 2, there was ultrasonically determining flow parameters of a fluid (tap water) flowing through a passage (painted steel pipe). The painted steel pipe had an inside diameter of 102 millimeters (mm), and a wall thickness of 7 millimeters (mm).

### Experimental Procedure

Example 2 was performed by using a similar ‘clamp-on’ type of experimental measuring and data acquisition system as the one used in Example 1, except for changes in the acoustic coupling material and minor changes in the detector array spacing and of the frequency of the transmitted ultrasound waves. The experimental measuring system was specially adapted and modified for ultrasonically determining flow parameters of tap water type of fluid flowing through a painted steel pipe type of passage, for implementing the method of the present invention (as illustratively described hereinabove, with reference to FIGS. 1-3).

In this example, epoxy glue was used as the coupling material between the transducers (both transmitter and detector arrays) and the painted steel pipe. The piezoceramic plates in the transmitter and detector arrays were not in direct contact with the pipe, but were separated by 5 mm thick perspex layers glued with epoxy to the piezoceramic plates. The spacing between the elements of the detector array was 1.5 mm and the ultrasound wave frequency was 5.0 MHz. In Example 2, the painted steel pipe was installed on an industrial facility for testing flow meters, and the flow rate was determined by electromagnetic type flow meters.

### Experimental Results

The results obtained for Example 2 are presented in FIGS. 5a and 5b, which are graphical presentations of exemplary results of implementing the method of the present invention, for ultrasonically determining flow parameters of tap water type of fluid flowing through a painted steel pipe type of passage. FIGS. 5a and 5b show the results of measurements acquired during 80 seconds for a constant tap water flow rate of 10.0±0.05 cubic meters per hour through the pipe.

FIG. 5a is a color map of a part of the logarithm of the filtered and normalized far-field scattering amplitude distribution, A(θ, Δf)/|Ψₜᵣₐₙₛ|, two-dimensional function, showing lines of integrals which were used to calculate the probability distribution function, P_{d}(v), in accordance with hereinabove described equations (11a, 11b, and 11c).

FIG. 5b is a graphical plot of the (extracted) probability distribution function, P_{d}(v), of the velocity v, plotted against velocity (v) [centimeters/second], that results from calculation of line integrals performed on the data presented in FIG. 5a. Negative values of P_{d}(v) can be interpreted as the presence of a net opposite direction of flow of the tap water through the pipe at a particular velocity (such presentation is convenient for calculating the flow rate of the tap water).

As described hereinabove, the second case of step (b) of the method of the present invention is based on, and includes, transforming (via using a Fourier transform procedure) the acquired near-field amplitude and phase change values (from Step (a)) expressed in terms of time series in the time domain, from the time domain to the frequency domain, and using the acquired near-field amplitude and phase change values expressed in terms of the frequency domain, for constructing the far-field scattering wave function in terms of the frequency domain.

Thus, in accordance to the second case of step (b) of the method of the present invention, for processing the results of Example 2, values of the measurements were initially transformed from the time domain to the frequency domain using a fast Fourier transform (FFT). The FFT bank size was 16,384 data points and the pulse repetition rate was about 1800 Hz, hence, the frequency shift scale of the data spanned between 0 to 900 Hz with a resolution of about 0.11 Hz. The higher the size of the FFT bank, the higher was the dynamical range of the flow rate measurement. The filtered far-field scattering amplitude distribution, A(θ, Δf), was calculated according to equation (7b).

The probability distribution function, P_{d}(v), was calculated from A(θ, Δf) according to equation (11b). The black lines over the color map in FIG. 5a demonstrate some of the lines integral chosen in calculation of the probability distribution function, P_{d}(v), according to equation (11b). Each section of line corresponds to a specific velocity, v, and is located along a line that intercepts the origin of the axes and spans between θ₁=0.75 degrees and θ₂=2.4 degrees of the scattering angle axis scale. The maximal velocity of P_{d}(v) was determined at a point vₘₐₓ, where all the values of equation (11c) that correspond to v>vₘₐₓ were lower than a threshold value. The threshold value was determined as the maximum between a value above the background noise at non-flow condition (0.001) and about ⅕ of a filtered highest value of equation (11c) at the specific flow rate.

In accordance with step (c) of the method of the present invention, the flow rate, Q, was calculated from the probability distribution function, P_{d}(v), according to equation (12), with the value of the statistical geometrical factor, G{P_{d}}, being G{P_{d}}=S. With reference to FIG. 5b, it is noted that negative values of P_{d}(v) are allowable in the calculation of the flow rate, Q, where such values represent an opposite (negative) part of the fluid flow when the integral is limited to values of the velocity, v, between v=0 and v=vₘₐₓ (based on use of the filtering procedure of equation (7b)). In the plot of FIG. 5b, for values of velocity, v, near v=0, values of P_{d}(v) become negative, possibly due to eddies (eddy currents) that contribute negative fluid flow, thus a small part of such negative flow is subtracted from the total flow rate, Q. The calculated flow rate, Q, was found to be 10.0 ±0.1 cubic meters per hour, exactly as expected.

Implementation of the method of the present invention was also successful using the same experimental setup and a similar experimental procedure (only varying the filtering procedure) for determining tap water flow rates in a range of between 0.5 and 270 cubic meters per hour.

The present invention, as illustratively described and exemplified hereinabove, has several beneficial and advantageous aspects, characteristics, and features, which are based on or/and a consequence of, the above illustratively described main aspects of novelty and inventiveness.

First, the present invention is generally applicable for ultrasonically determining flow parameters (particularly, peak velocity, velocity distribution, and flow rate) of essentially any type or kind, and form, of fluid (liquid or/and gas) flowing through essentially any type or kind, and size, of passage (channel, conduit, or duct) of essentially any type or kind, and size (small scale, medium scale, large scale), of process.

Second, the present invention is generally applicable to a homogeneous or inhomogeneous, single phase or multiple phase, particulate-free or particulate-containing, liquid, such as water, an organic solvent, or a petroleum based liquid, flowing through a passage (e.g., pipe, tube) of a medium or large scale process (e.g., a residential or commercial clean water or waste water distribution process, an industrial manufacturing process, or a petroleum based liquid transfer process), or, such as a biological liquid (e.g., blood, urine, water), flowing through a passage (e.g., vessel, duct, organ) of a small scale biological (e.g., human or animal) process.

Third, the inventive method is generally applicable to a turbulent flowing fluid or a laminar flowing fluid (i.e., characterized by a high or low Reynolds number, respectively).

Fourth, the inventive method is generally implementable by using various different types of equipment and hardware, and associated software, which are known for ultrasonically determining flow parameters of a fluid flowing through a passage. The inventive method is particularly implementable by using ‘clamp-on’ types of equipment and hardware, and associated software, which include an ultrasound wave transmitter and an ultrasound wave detector array, clamped on, in an oppositely facing configuration, to outer walls of a passage through which the fluid flows, and which operate by transmitting and detecting, respectively, ultrasound waves that propagate normal or perpendicular to the main or net flow direction of the flowing fluid, and are scattered by the flowing fluid.

Fifth, in view of the preceding beneficial and advantageous aspects, characteristics, and features, the present invention is readily commercially applicable to a wide variety of different fields and areas of industry which require, or would benefit from, determination of flow parameters (particularly, peak velocity, velocity distribution, and flow rate) of a fluid flowing through a passage.

Based upon the above indicated aspects of novelty and inventiveness, and, beneficial and advantageous aspects, characteristics, and features, the present invention successfully overcomes shortcomings and limitations, and widens the scope, of presently known techniques in the field(s) encompassing or/and relating to ultrasonically determining flow parameters (particularly, peak velocity, velocity distribution, and flow rate) of a fluid (liquid or/and gas) flowing through a passage (channel, conduit, or duct).

It is appreciated that certain aspects and characteristics of the invention, which are, for clarity, described in the context of separate embodiments, may also be provided in combination in a single embodiment. Conversely, various aspects and characteristics of the invention, which are, for brevity, described in the context of a single embodiment, may also be provided separately or in any suitable sub-combination.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

While the invention has been described in conjunction with specific embodiments and examples thereof, it is evident that many alternatives, modifications and variations will be apparent to those skilled in the art. Accordingly, it is intended to embrace all such alternatives, modifications and variations that fall within the scope of the appended claims.

### REFERENCES

1. Papadakis, E. E., Ultrasonic Instruments and Devices, Academic Press, New York (2000).
2. U.S. Pat. No. 7,069,776, to Ishikawa, et al., entitled: “Ultrasonic Flow Meter And Ultrasonic Sensor”.
3. U.S. Pat. No. 7,000,485, to Ao, et al., entitled: “Flow Measurement System With Reduced Noise And Crosstalk.
4. U.S. Pat. No. 6,907,792, to Ohnishi, entitled: “Method For Measuring Flow Of Fluid Moving In Pipe Or Groove-like Flow Passage”.
5. U.S. Pat. No. 6,907,361, to Molenaar, et al., entitled: “Ultrasonic Flow-measuring Method”.
6. U.S. Pat. No. 6,732,595, to Lynnworth, entitled: “Method Of And System For Determining The Mass Flow Rate Of A Fluid Flowing In A Conduit”.
7. U.S. Pat. No. 6,626,049, to Ao, et al., entitled: “Clamp-on Steam/gas Flow Meter”.
8. U.S. Pat. No. 6,463,808, to Hammond, entitled: “Ultrasonic Measurement System With Chordal Path”.
9. U.S. Pat. No. 6,089,104, to Chang, entitled: “Ultrasonic Flow Meter Using Transit Time Across Tube Chords For Determining The Flow Rates”.
10. U.S. Pat. No. 5,719,329, to Jepson, et al., entitled: “Ultrasonic Measuring System And Method Of Operation”.
11. Worch, A., “A Clamp-on Ultrasonic Cross Correlation Flow Meter For One-phase Flow”, Meas. Sci. Technol., 9: 622-630 (1998).
12. U.S. Pat. No. 6,826,965, to Liu, entitled: “Anti-parallel Tag Flow Measurement System”.
13. U.S. Pat. No. 6,502,465, to Vedapuri, et al., entitled: “Determining gas and liquid flow rates in a multi-phase flow”.
14. U.S. Pat. No. 7,077,012, to Hirayama, et al., entitled: “Wedge And Wedge Unit For Use In Ultrasonic Doppler Flow Meter”.
15. U.S. Pat. No. 6,758,100, to Huang, entitled: “Doppler Flowmeter For Multiphase Flows”.
16. U.S. Pat. No. 6,067,861, to Shekarriz, et al., entitled: “Method And Apparatus For Ultrasonic Doppler Velocimetry Using Speed Of Sound And Reflection Mode Pulsed Wideband Doppler”.
17. U.S. Pat. No. 4,391,149, to Herzl, entitled: “Doppler-type Ultrasonic Flowmeter”.
18. U.S. Pat. No. 4,333,353, to Baumoel, entitled: “Two-transducer Doppler Flowmeter With Swept Oscillator”.
19. U.S. Pat. No. 4,208,908, to Hickox, entitled: “Speed Of Sound Compensation For Doppler Flowmeter”.
20. Seifer, Sh., and Steinberg, V., “Flow Induced Ultrasound Scattering: Experimental Studies”, Physics of Fluids, 16(5): 1587-1602 (May, 2004).
21. Seifer, Sh., and Steinberg, V., “Spatial And Temporal Turbulent Velocity And Vorticity Power Spectra From Sound Scattering”, Physical Review E 71, 045601(R) (2005).
22. Fabrikant, A. L., “Sound Scattering By Vortex Flows”, Soy. Phys. Acoust., 29(2): 152-154 (1983).
23. Lund, F., and Rojas, C., “Ultrasound As A Probe Of Turbulence”, Physica D, 37: 508-514 (1989).
24. P. Tortoli, G. Guidi, F. Guidi, C. Atzeni, “A Review Of Experimental Transverse Doppler Studies”, IEEE Trans. Ultrasonics, Ferroelectrics, and Frequency Control, 41 (1): 84-89 (1994).
25. D. Censor, V. L. Newhouse, T. Vontz, “Theory Of Ultrasound Doppler Spectra Velocimetry For Arbitrary Beam And Flow Configuration”, IEEE Trans. Biomed. Eng., vol. BME-35, no. 9, 740-751 (1988).
26. I. H. Shames, “Mechanics of Fluids”, McGraw-Hill (2002).

1. A method for ultrasonically determining flow parameters of a fluid flowing through a passage, by using far-field analysis, the method comprising:
(a) acquiring near-field amplitude and phase change values of ultrasound waves transmitted into, propagating through, and scattered by, the flowing fluid;
(b) determining a far-field scattering amplitude distribution, as a two-dimensional function of scattering angle and Doppler frequency shift, from said acquired near-field amplitude and phase change values; and
(c) determining the flow parameters of the flowing fluid, from said far-field scattering amplitude distribution.

2. The method of claim 1, wherein said near-field amplitude and phase change values are acquired for a measuring plane defined by, and including, a same plane of a transmitting region or zone of an ultrasound wave transmitter device and a detecting region or zone of an ultrasound wave detector array device, wherein direction of said transmitted ultrasound waves is normal or perpendicular to main or net direction of flow of the flowing fluid.

3. The method of claim 1, wherein said near-field amplitude and phase change values are acquired in a near-field region or zone characterized and defined by a near-field distance extending or spanning from (i) a position or location of a scatterer located within a scattering region or zone of the flowing fluid subjected to, and scattering, said ultrasound waves transmitted into, and propagating through, the flowing fluid by an ultrasound wave transmitter device, until (ii) a position or location of a detecting region or zone of an ultrasound wave detector array device detecting said scattered ultrasound waves.

4. The method of claim 3, wherein said near-field region or zone is characterized and defined by relation or condition: b_{NF}≦d²/2λ, wherein said parameter b_{NF} is said near-field distance, said parameter d is smaller length of either (i) length of transmitting region or zone of said ultrasound wave transmitter device or (ii) length of said detecting region or zone of said ultrasound wave detector array device, and said parameter λ is wavelength of said transmitted or scattered ultrasound waves.

5. The method of claim 3, wherein said near-field distance is evaluated for a measuring plane defined by, and including, a same plane of a transmitting region or zone of said ultrasound wave transmitter device and said detecting region or zone, wherein direction of said transmitted ultrasound waves is normal or perpendicular to main or net direction of flow of the flowing fluid.

6. The method of claim 3, wherein said scatterer is a feature or characteristic of, or within, the flowing fluid which scatters said transmitted ultrasonic waves propagating through the flowing fluid, thereby causing a change in velocity of said transmitted ultrasonic waves compared to velocity of said transmitted ultrasonic waves propagating through the flowing fluid which are not scattered by said feature or characteristic.

7. The method of claim 6, wherein said feature or characteristic is an internally existing, or/and externally caused, flow fluctuation arising due to a velocity, pressure, thermal, concentration, or/and density inhomogeneity or gradient, or/and turbulence, moving in the flowing fluid.

8. The method of claim 6, wherein said feature or characteristic is an internally existing, or/and externally provided, substance moving in the flowing fluid.

9-10. (canceled)

11. The method of claim 1, wherein said ultrasound waves have a frequency in a range of between about 20,000 cycles per second (20 kHz or 0.02 MHz) and about 20,000,000 cycles per second (20,000 kHz or 20 MHz).

12. (canceled)

13. The method of claim 1, wherein said transmitted ultrasound waves are transmitted into the flowing fluid by a clamp-on type of ultrasound wave transmitter assembly, clamped onto an outside surface of the passage in a configuration such that said transmitted ultrasound waves are transmitted into the flowing fluid in a direction normal or perpendicular to main or net flow direction of the flowing fluid.

14. The method of claim 1, wherein said ultrasound waves are detected by a clamp-on type of ultrasound wave detector array assembly, clamped onto an outside surface of the passage in a configuration such that said detected ultrasound waves are detected in a direction normal or perpendicular to main or net flow direction of the flowing fluid.

15. The method of claim 1, wherein said ultrasound waves are detected by a clamp-on type of ultrasound wave detector array assembly, clamped onto an outside surface of the passage in a configuration oppositely facing, and aligned with, an ultrasound wave transmitter assembly, in a measuring plane defined by, and including, a transmitting region or zone of said ultrasound wave transmitter assembly and a detecting region or zone of said ultrasound wave detector array assembly.

16. (canceled)

17. The method of claim 1, wherein said near-field amplitude and phase change values are acquired according to a heterodyne type scheme of data acquisition, or a direct analog to digital conversion type scheme of data acquisition.

18. (canceled)

19. The method of claim 1, wherein said far-field scattering amplitude distribution is determined in a far-field region or zone characterized and defined by a far-field distance extending or spanning from (i) a position or location of a scatterer located within a scattering region or zone of the flowing fluid subjected to, and scattering, said ultrasound waves transmitted into, and propagating through, the flowing fluid by an ultrasound wave transmitter device, until (ii) a position or location located at or beyond a detecting region or zone of an ultrasound wave detector array device detecting said scattered ultrasound waves.

20. The method of claim 19, wherein said far-field region or zone is characterized and defined by relation or condition: b_{FF}>>d²/2λ, wherein said parameter b_{FF} is said far-field distance, said parameter d is smaller length of either (i) length of transmitting region or zone of said ultrasound wave transmitter device or (ii) length of said detecting region or zone of said ultrasound wave detector array device, and said parameter 2 is wavelength of said transmitted or scattered ultrasound waves.

21. The method of claim 1, wherein said far-field scattering amplitude distribution is determined for a far-field virtual propagation of said ultrasound waves transmitted into, propagating through, and scattered by, the flowing fluid, being represented by far-field virtual transmitted ultrasound waves, and far-field virtual scattered ultrasound waves, virtually propagating in a direction of a far-field virtual position or location located at a far-field virtual distance, at or beyond a detecting region or zone of an ultrasound wave detector array assembly detecting said scattered ultrasound waves.

22. (canceled)

23. The method of claim 1, wherein step (b) is performed according to either a first case, based on using said acquired near-field amplitude and phase change values expressed in terms of time series in a time domain, or, a second case, based on using said acquired near-field amplitude and phase change values expressed in terms of frequency components in a frequency domain, wherein said first case and said second case differ according to order of using a Fourier transform procedure.

24. The method of claim 1, wherein step (b) includes constructing a far-field scattering wave function in terms of a near-field scattering wave function, from said acquired near-field amplitude and phase change values.

25. (canceled)

26. The method of claim 24, wherein said far-field scattering wave function is based on application of a mathematical description of Huygens' Principle of optics to said acquired near-field amplitude and phase change values.

27. The method of claim 1, wherein step (b) is based on, and includes, using said acquired near-field amplitude and phase change values expressed in terms of time series in a time domain, for constructing a far-field scattering wave function in terms of said time domain, which is then transformed, via using a Fourier transform procedure, from said time domain to a frequency domain.

28. (canceled)

29. The method of claim 1, wherein step (b) is based on, and includes, transforming, via using a Fourier transform procedure, said acquired near-field amplitude and phase change values expressed in terms of time series in a time domain, from said time domain to a frequency domain, and using said acquired near-field amplitude and phase change values expressed in terms of said frequency domain, for constructing a far-field scattering wave function in terms of said frequency domain.

30-31. (canceled)

32. The method of claim 1, wherein step (c) includes determining a peak velocity of the flowing fluid, being value of velocity of the flowing fluid corresponding to a peak in distribution function of velocity component of the flowing fluid which is normal or perpendicular to direction of said transmitted ultrasound waves.

33. The method of claim 32, wherein said peak velocity is defined by, and determined from, an equation including a term for a partial derivative of said Doppler frequency shift with respect to said scattering angle, and a term for wavenumber of said transmitted ultrasound waves, corresponding to a slope, in terms of an axis of said Doppler frequency shift, with respect to an axis of said scattering angle, of a best fitting line of a crest, or crest-like, shape or form, in a graphical plot of said far-field scattering amplitude distribution two-dimensional function.

34. (canceled)

35. The method of claim 34, wherein said equation corresponds to direction of said crest, or crest-like, shape or form, which is visually observable in said graphical plot of said far-field scattering amplitude distribution two-dimensional function.

36. The method of claim 32, wherein said peak velocity is used for determining a velocity distribution of the flowing fluid.

37. The method of claim 36, wherein said velocity distribution is determined in terms of a probability distribution function of said velocity component of the flowing fluid which is normal or perpendicular to direction of said transmitted ultrasound waves.

38. The method of claim 33, wherein said crest, or crest-like, shape or form, in said graphical plot of said far-field scattering amplitude distribution is used for determining a velocity distribution of the flowing fluid.

39. The method of claim 37, wherein said peak velocity and said probability distribution function are used for determining a flow rate of the flowing fluid.

40. The method of claim 37, wherein flow rate of the flowing fluid is determined in terms of (i) said peak velocity, (ii) said probability distribution function, (iii) value of cross-sectional area of the passage through which flows the fluid, and (iv) a statistical geometrical factor representing a function of geometrical characteristics and parameters relating to the flowing fluid, the passage, transmission of said ultrasound waves into the flowing fluid, and measurement of said scattered ultrasound waves.

41-46. (canceled)

47. The method of claim 1, wherein the passage is a vessel, duct, or organ, of a small scale biological process, and the fluid is a biological liquid.
